(19) United States
(12) Patent Application Publication
(10) Pub. No.: US 2025/0352663 A1
(43) Pub. Date: Nov. 20, 2025

(54) Nanoclusters Functionalized with Adenosine Triphosphate or an Analogue and Their Use

(71) Applicant: The Board of Trustees of the Leland Stanford Junior University, Stanford, CA (US)

(72) Inventors: Laurent Bekale, Redwood City, CA (US); Peter Luke Santa Maria, Emerald Hills, CA (US)

(21) Appl. No.: 19/101,503

(22) PCT Filed: Aug. 23, 2023
(86) PCT No.: PCT/US2023/072735

§ 371 Date: Feb. 5, 2025

Related U.S. Application Data
(60) Provisional application No. 63/373,409, filed on Aug. 24, 2022.

(51) Int. Cl. A61K 47/69; A61K 31/7076; A61K 45/06; A61K 47/52; A61P 31/04; A61P 35/00; B82Y 5/00

(52) U.S. Cl. CPC A61K 47/6923; A61K 31/7076; A61K 45/06; A61K 47/52; A61K 47/6929; A61P 31/04; A61P 35/00; B82Y 5/00

(57) ABSTRACT

Compositions, methods, and kits are provided for treating infections and cancer with metallic nanoclusters. In particular, metallic nanoclusters having a size of less than 10 nm that are conjugated to adenosine triphosphate (ATP) or an analogue thereof can be used to eradicate a cell in a growth arrest phase such as infectious bacterial or fungal cells. Such nanoclusters can also induce endoplasmic reticulum stress and inhibit growth of cancerous cells. Additionally, such metallic nanoclusters can be used to inhibit a purinergic P2X7 receptor and FtsH protease.

### CROSS REFERENCE TO RELATED APPLICATION

This application claims benefit of U.S. Provisional Patent Application No. 63/373,409, filed Aug. 24, 2022, which application is incorporated herein by reference in its entirety.

### STATEMENT REGARDING FEDERALLY SPONSORED RESEARCH OR DEVELOPMENT

This invention was made with Government support under contract Al154097 awarded by the National Institutes of Health. The Government has certain rights in the invention.

### BACKGROUND

Antibiotics are the mainstay of modern clinical medicine. However, bacteria develop resistance to both natural and synthetic antibiotics within years of their first clinical use (Walsh (2003) Nature Reviews Microbiology 1:65-70). Current mechanisms of antibiotic resistance include: decreased uptake by changes in outer membrane permeability; antibiotic excretion by activation of efflux pump-proteins; enzymatic modification of the antibiotic; modification of antibiotic targets; and bacterial physiology such as biofilm (van Hoek et al. (2011) Front. Microbiol. 2:203).

In the United States and Europe alone, over 50,000 people die every year because of resistant infections (The Review on Antimicrobial Resistance. Antimicrobial Resistance: Tackling a crisis for the health and wealth of nations (2014), amr-review.org/Publications.html)). Lengths of stays in a hospital are prolonged by antibiotic-resistant infections, and these same infections are often acquired in hospitals. The economic impact of antibiotic resistant infections is estimated to be between US $5 billion and US $24 billion per year in the United States alone (Hall (2004) Nature Reviews Microbiology 2:430-435). However, the drug pipelines of pharmaceutical companies have not kept pace with the evolution of antibiotic resistance. In 2004, only 1.5% of all the drugs in development by the world's 15 largest pharmaceutical companies were antibiotics (Smith and Coast, “The economic burden of antimicrobial resistance: why it is more serious than current studies suggest.” (2012), researchgate.net/publication/291413454). The new reality that we must face is that the pharmaceutical companies are not presently aligned for the discovery of new antibiotics. A strategy to protect our existing antibiotics is through the use of antibiotic adjuvants, compounds that enhance the activity of current drugs and minimize, and even directly block resistance (Lu et al. (2009) Proc. Natl. Acad. Sci. U.S.A. 106(12):4629-4634, Gonzalez-Bello (2017) Bioorg. Med. Chem. Lett. 27(18):4221-4228). Another strategy is the used of ant-virulence agents. These agents can circumvent antibiotic resistance by disarming pathogens of virulence factors that facilitate human disease while leaving bacterial growth pathways (Dickey et al. (2017) Nat. Rev. Drug Discov. 16(7):457-471).

Bacterial cells, attached to a surface, can aggregate to each other to form biofilms. Bacteria growing biofilms may exhibit increased tolerance to antimicrobial agents, it is very difficult or eliminate substantially reduce. Biofilm bacteria have two dormant phenotypes: the viable but non-culturable (VBNC) state and the persister state. Dormant phenotypes (VBNC and persisters) allow bacteria to survive in conditions that are deadly to the rest of their genetically identical lineage. Once in biofilms, they can escape the immune system. Thus, one of the main roles of biofilm is to provide a protective habitat for persisters and VBNC by shielding them from the immune system (Lewis (2010) Microbe (Washington, D.C.) 5(10):429-437). Another property of biofilms is their capacity to be more resistant to antimicrobial agents than planktonic cells (Spoering et al. (2001) J. Bacteriol. 183(23):6746-6751). Thus, there is an ongoing and unmet need for an improved approach to treating antibiotic resistant infections.

### SUMMARY

Compositions, methods, and kits are provided for treating infections and cancer with metallic nanoclusters. In particular, metallic nanoclusters having a size of less than 10 nm that are conjugated to adenosine triphosphate (ATP) or an analogue thereof can be used to eradicate a cell in a growth arrest phase such as infectious bacterial or fungal cells. Such nanoclusters can also induce endoplasmic reticulum stress and inhibit growth of cancerous cells. Additionally, such metallic nanoclusters can be used to inhibit a purinergic P2X7 receptor and FtsH protease.

In one aspect, a method of eradicating a cell in a growth arrest phase is provided, the method comprising contacting the cell in the growth arrest phase with an effective amount of a metallic nanocluster having a size of less than 10 nm, wherein the nanocluster is conjugated to adenosine triphosphate (ATP) or an analogue thereof.

In certain embodiments, the cell is a prokaryotic cell or a eukaryotic cell. In some embodiments, the cell is a bacterial cell, fungal cell, or a human cell. In some embodiments, the cell is a benign tumor cell or a malignant tumor cell.

In certain embodiments, the nanocluster has a diameter of less than 5 nm. In some embodiments, the diameter ranges from about 1 nm to about 5 nm, including any diameter within this range such as 1.0 nm, 1.5 nm, 2.0 nm, 2.5 nm, 3.0 nm, 3.5 nm, 4.0 nm, 4.5 nm, or 5 nm. In some embodiments, the diameter is about 2 nm.

In certain embodiments, the ATP analogue is selected from the group consisting of ATPαS, ATPβS, ATPγS, deoxyadenosine triphosphate (dATP), 7-deazaadenosine-5′-triphosphate (7-deaza-ATP, and β,γ-methyleneadenosine 5′-triphosphate (AMP-PCP).

In certain embodiments, the metallic nanocluster comprises a noble metal. In some embodiments, the noble metal is gold.

In certain embodiments, the nanocluster is conjugated to at least 1000 ATP molecules.

In another aspect, a composition for use in a method of treating an infection by bacteria or fungi in a growth arrest phase is provided, the composition comprising a metallic nanocluster having a size of less than 10 nm, wherein the nanocluster is conjugated to adenosine triphosphate (ATP) or an analogue thereof.

In certain embodiments, the composition further comprises a pharmaceutically acceptable excipient or carrier.

In certain embodiments, the composition further comprises an antibiotic or an antifungal agent.

In another aspect, a method of treating a subject for an infection by bacteria or fungi in a growth arrest phase is provided, the method comprising administering a therapeutically effective amount of a composition comprising a metallic nanocluster having a size of less than 10 nm, wherein the nanocluster is conjugated to adenosine triphosphate (ATP) or an analogue thereof to the subject.

In certain embodiments, the infection is a chronic bacterial or fungal infection such as, but not limited to, tuberculosis, cystic fibrosis, cutaneous wound infections, urinary tract infections or a biofilm associated infections, including, but not limited to catheter-associated infections, central line-associated infections, endotracheal tube-associated infections, implantable devices-associated infections including prosthetic joint infections.

In certain embodiments, the composition is administered locally at the site of infected tissue. For example, for an ear infection, the composition may be administered locally into the ear canal.

In certain embodiments, the method further comprises administering a therapeutically effective amount of at least one antibiotic or antifungal agent to the subject.

In certain embodiments, multiple cycles of treatment are administered to the subject.

In certain embodiments, the bacteria are Gram-negative bacteria.

In another aspect, a method of treating cancer in a subject is provided, the method comprising administering a therapeutically effective amount of a composition comprising a metallic nanocluster having a size of less than 10 nm, wherein the nanocluster is conjugated to adenosine triphosphate (ATP) or an analogue thereof to the subject.

In certain embodiments, the composition is administered locally, intratumorally, intravenously, subcutaneously, by inhalation, or topically. In some embodiments, the composition is administered locally to a tumor.

In certain embodiments, the multiple cycles of treatment are administered to the subject.

In certain embodiments, the cancer is melanoma or schwannoma.

In another embodiment, a method of treating melanoma in a subject is provided, the method comprising administering to the subject a therapeutically effective amount of ATP in combination with a therapeutically effective amount of a metallic nanocluster having a size of less than 10 nm, wherein the nanocluster is conjugated to adenosine triphosphate (ATP) or an analogue thereof.

In another aspect, a composition for use in a method of treating cancer is provided, the composition comprising a metallic nanocluster having a size of less than 10 nm, wherein the nanocluster is conjugated to adenosine triphosphate (ATP) or an analogue thereof. In certain embodiments, the cancer is melanoma or schwannoma.

In certain embodiments, the composition further comprises a pharmaceutically acceptable excipient or carrier.

In certain embodiments, the composition further comprises an anti-cancer agent.

In another aspect, a method of inhibiting a FtsH protease is provided, the method comprising contacting the FtsH protease with a metallic nanocluster having a size of less than 10 nm, wherein the nanocluster is conjugated to adenosine triphosphate (ATP) or an analogue thereof, wherein the protease activity of the FtsH protease is inhibited.

In another aspect, a method of inhibiting a purinergic P2X7 receptor (P2X7R) is provided, the method comprising contacting the P2X7R with a metallic nanocluster having a size of less than 10 nm, wherein the nanocluster is conjugated to adenosine triphosphate (ATP) or an analogue thereof, wherein the activity of the P2X7R is inhibited.

In another aspect, a method of increasing phagocytic clearance in a tissue is provided, the method comprising contacting the tissue with a metallic nanocluster having a size of less than 10 nm, wherein the nanocluster is conjugated to adenosine triphosphate (ATP) or an analogue thereof, wherein the phagocytic clearance is increased in the tissue.

In another aspect, a method of reducing NLRP3 activation and IL-1beta-mediated inflammation in a subject is provided, the method comprising administering a therapeutically effective amount of a metallic nanocluster having a size of less than 10 nm to the subject, wherein the nanocluster is conjugated to adenosine triphosphate (ATP) or an analogue thereof. The therapeutically effective amount of the metallic nanocluster may also reduce microglial inflammation, reduces oxidative stress, and increases phagocytic clearance in the subject.

In another aspect, a method of inducing endoplasmic reticulum (ER) stress in a cell is provided, the method comprising contacting the cell with an effective amount of a metallic nanocluster having a size of less than 10 nm, wherein the nanocluster is conjugated to adenosine triphosphate (ATP) or an analogue thereof.

In another aspect, a method of inhibiting proliferation of a cancerous cell is provided, the method comprising contacting the cell with an effective amount of a metallic nanocluster having a size of less than 10 nm, wherein the nanocluster is conjugated to adenosine triphosphate (ATP) or an analogue thereof.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. Influence of ATP-mediated P2X7R activation on tumor and immune system cells (Grassi et al. (2021) Front. Cell Dev. Biol. 9:694831).

FIG. 2. Proposed pathways of ATP and adenosine before, during, and after continuous intravenous infusions of ATP in humans. The primary extracellular ATP (eATP) source is the infused ATP, which delivers ATP directly into the blood plasma compartment. After a short infusion time, the elevated eATP pools induce higher activities of catabolic ecto-enzymes. At the termination of 8 h continuous intravenous infusion of ATP, most of the exogenously administered ATP is sequestered in the erythrocytes, and all the eATP has been degraded. Blood samples were collected from the arm contralateral to the infusion arm. Intravenous infusions of ATP were at 50-100 μg/kg min of ATP (Rapaport et al. (2015) Purinergic Signal 11(2):251-62).

FIGS. 3A-3D. Characterizations of AuNC@ATP. The suspension color of AuNC@ATP (1 mg/ml) in PBS is yellowish, not pinkish or purple (FIG. 3A), indicating that the diameter of AuNC@ATP is less than 4 nm (PMID: 28426435). From the UV-vis spectrum, we can observe a monotonous decrease from 450 nm to 700 nm but no surface plasmon resonance peak at 520 nm (FIG. 3B). Transmission electron microscopy (TEM) analysis confirms that the size of AuNC@ATP is ultrasmall 2.45±0.43 nm (FIGS. 3C and 3D). The AuNC@ATP is negatively charged at pH 7.4, with a Z-potential value of −30±2 mV in water.

FIG. 4. A schematic view of P2X7 receptor. Top left: Topology of the receptor. Top right: Top view of the receptor. Bottom left: Side view of the receptor showing the extracellular, intracellular and transmembrane domains. Agonist binding sites are presumably located at subunit interfaces in the extracellular side of the receptor. Bottom right: Longitudinal cross-section of the receptor, showing the pore domain.

FIGS. 5A-5E. Effect of AuNC@ATP antagonist of P2X7 on ATP-induced ethidium uptake in J774A-1 macrophages. The activation of the P2X7 receptor with high concentrations of adenosine triphosphate (ATP>0.05 mM) results in the formation of a large transmembrane pore permeable to molecules up to 900 Da such as Ethidium bromide (EtBr). Therefore, the inhibition of the P2X7 activation (i.e., pore formation) was quantified as the reduction of ATP-induced ethidium uptake in J774A-1 macrophages expressing. The EtBr uptake was induced in J774A-1 by adding ATP (2 mM). The antagonists of the P2X7 receptor (i.e., O-ATP and AuNC@ATP) and ATP were added at the same time. Then cells were incubated for 1 h at 37° C. before measuring the fluorescence of ethidium bromide. FIG. 5A shows P2X7 transmembrane pore formation measured by ethidium bromide uptake by J774A-1 macrophages. FIG. 5B shows the percent of the control response for ATP-induced ethidium bromide uptake in the presence of ATP, ATP+0-ATP, or ATP+AuNC@ATP. FIG. 5C shows the fluorescence spectrum for J774A-1 cells incubated with ethidium bromide, ATP, ATP+ethidium bromide, 2× ATP+o-ATP+ethidium bromide, 2× ATP+AuNC@ATP+ethidium bromide, or ATP+AuNC@ATP+ethidium bromide. FIG. 5D shows a schematic illustrating formation of a large transmembrane pore upon activation of the P2X7 receptor with adenosine triphosphate. FIG. 5E shows the chemical formula of ethidium bromide and intercalation of ethidium bromide into DNA.

FIG. 6. AuNC@ATP strongly inhibits the P2X7R and may potentially inhibit this pathway for a multitude of diseases.

FIG. 7. P2X7 receptor mediates NLRP3 inflammasome activation. Patients with the p.Ser624Arg NLRP3 mutation present increased NLRP3 activity ex vivo.

FIGS. 8A-8C. Bioluminescent ATP assay.

FIGS. 9A-9B. AuNC@ATP induces cell death and ER stress in schwannoma cells. (FIG. 9A) The AuNC@ATP induced a concentration-dependent cell viability diminution in schwannoma cells. (FIG. 9B) Fluorescence spectrum of Th-T observed in schwannoma cells treated with various concentrations of AuNC@ATP.

FIGS. 10A-10C. AuNC@ATP slows the growth of melanoma cells and hinders tumor development in a xenograft model. (FIG. 10A) The AuNC@ATP induced a concentration-dependent cell viability diminution in B16-F10 melanoma cells. (FIG. 10B) The Illustration shows how AuNC@ATP inhibits B16-F10 cell proliferation and compares cell growth rates in the absence and presence of eATP, AuNC@ATP, and their combination. (FIG. 10C) Image displaying the tumor weight of mice with B16 melanoma who received PBS, eATP, and AuNC@ATP+eATP treatments. The graph shows a comparison of the tumor weight of the mice on day 14 after treatment.

### DETAILED DESCRIPTION OF EMBODIMENTS

Compositions, methods, and kits are provided for treating infections and cancer with metallic nanoclusters. In particular, metallic nanoclusters having a size of less than 10 nm that are conjugated to adenosine triphosphate (ATP) or an analogue thereof can be used to eradicate a cell in a growth arrest phase such as infectious bacterial or fungal cells. Such nanoclusters can also induce endoplasmic reticulum stress and inhibit growth of cancerous cells. Additionally, such metallic nanoclusters can be used to inhibit a purinergic P2X7 receptor and FtsH protease.

Before the present compositions comprising nanoclusters conjugated to ATP and methods of using them are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a bacterial cell” includes a plurality of such bacterial cells and reference to “the nanocluster” includes reference to one or more nanoclusters and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “nanocluster” refers to a metallic nanocluster having a size ranging from about 0.5 nm to about 10 nm in length. Nanoclusters may have dimensions of 10 nm or less, including 9 nm or less, or 8 nm or less, or 7 nm or less, or 6 nm or less, or 5 nm or less, or 4 nm or less, or 3 nm or less, or 2.5 nm or less, or 2.0 nm or less, or 1.5 nm or less, or 1.0 nm or less. In some instances, the nanocluster has dimensions ranging from 1 nm to 5 nm in length, including any length within this range such as 1 nm, 1.5 nm, 2.0 nm, 2.5 nm, 3.0 nm, 3.5 nm, 4.0 nm, 4.5 nm, or 5 nm in length.

“Diameter” as used in reference to a shaped structure (e.g., nanocluster) refers to a length that is representative of the overall size of the structure. The length may in general be approximated by the diameter of a circle or sphere that circumscribes the structure.

The term “persister cells” refers to cells that have entered a non-growing (i.e., dormant) or extremely slow-growing physiological state that renders them less susceptible or resistant to antimicrobial drugs. Such cells may “persist” after planktonic bacterial cells have been eradicated by the immune system or conventional treatment with an antimicrobial agent. Persister cells are commonly found in biofilms.

The terms “tumor,” “cancer” and “neoplasia” are used interchangeably and refer to a cell or population of cells whose growth, proliferation or survival is greater than growth, proliferation or survival of a normal counterpart cell, e.g., a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma, and include cancers such as, but are not limited to, pancreatic cancer, lung cancer (non-small cell lung cancer, small cell lung cancer), gastric cancer, ovarian cancer, endometrial cancer, colorectal cancer, oral cancer, skin cancer, cholangiocarcinoma, head and neck cancer, breast cancer, ovarian cancer, melanoma, peripheral neuroma, glioblastoma, adrenocortical carcinoma, AIDS-related lymphoma, anal cancer, bladder cancer, meningioma, glioma, astrocytoma, cervical cancer, chronic myeloproliferative disorders, colon cancer, endometrial cancer, ependymoma, esophageal cancer, Ewing's sarcoma, extracranial germ cell tumors, extrahepatic bile duct cancer, gallbladder cancer, gastrointestinal carcinoid tumors, gestational trophoblastic tumors, hairy cell leukemia, Hodgkin lymphoma, non-Hodgkin lymphoma, hypopharyngeal cancer, islet cell carcinoma, Kaposi sarcoma, laryngeal cancer, leukemia, lip cancer, oral cavity cancer, liver cancer, malignant mesothelioma, medulloblastoma, Merkel cell carcinoma, metastatic squamous neck cell carcinoma, multiple myeloma and other plasma cell neoplasms, mycosis fungoides and the Sezary syndrome, myelodysplastic syndromes, nasopharyngeal cancer, neuroblastoma, oropharyngeal cancer, bone cancers, including osteosarcoma and malignant fibrous histiocytoma of bone, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, small intestine cancer, soft tissue sarcoma, supratentorial primitive neuroectodermal tumors, schwannoma, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors.

As used herein, the term “antimicrobial agent” is interchangeable with the term “antibiotic” and refers to any agent capable of having bactericidal or bacterial static effects on growth. Antibiotics include, but are not limited to, a β-lactam antibiotic, an aminoglycoside, an aminocyclitol, a quinolone, a tetracycline, a macrolide, a lincosamide, a glycopeptide, a lipopeptide, a polypeptide antibiotic, a sulfonamide, trimethoprim, chloramphenicol, isoniazid, a nitroimidazole, a rifampicin, a nitrofuran, methenamine, and mupirocin.

The term “anti-bacterial effect” means the killing of, or inhibition or stoppage of the growth and/or reproduction of bacteria.

The term “anti-fungal effect” means the killing of, or inhibition or stoppage of the growth and/or reproduction of fungi.

By “anti-tumor effect” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

A “therapeutically effective amount” is intended for an amount of an active agent which is necessary to impart therapeutic benefit to a subject. For example, a “therapeutically effective amount” is an amount which induces, ameliorates or otherwise causes an improvement in the pathological symptoms, disease progression or physiological conditions associated with a disease, or which improves resistance to a disorder. Additionally, a “therapeutically effective amount” is an amount that can eradicate a cell in a growth arrest phase. In some cases, a “therapeutically effective amount” is an amount having an anti-bacterial, anti-fungal, or anti-tumor effect.

By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models, such as xenograft models of human renal cell carcinoma. See, e.g., Pulkkanen et al., In Vivo (2000) 14:393-400 and Everitt et al., Toxicol. Lett. (1995) 82-83:621-625 for a description of animal models.

The term “tumor response” as used herein means a reduction or elimination of all measurable lesions. The criteria for tumor response are based on the WHO Reporting Criteria [WHO Offset Publication, 48-World Health Organization, Geneva, Switzerland, (1979)]. Ideally, all uni- or bidimensionally measurable lesions should be measured at each assessment. When multiple lesions are present in any organ, such measurements may not be possible and, under such circumstances, up to 6 representative lesions should be selected, if available.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

“Substantially purified” generally refers to isolation of a component such as a substance (compound, nanoparticle, nucleic acid, polynucleotide, RNA, DNA, protein, or polypeptide) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography, gel filtration, and sedimentation according to density.

The terms “recipient”, “individual”, “subject”, “host”, and “patient”, are used interchangeably herein and refer to any vertebrate subject for whom diagnosis, treatment, or therapy is desired, particularly humans. By “vertebrate subject” is meant any member of the subphylum Chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

“Biocompatible” generally refers to a material and any metabolites or degradation products thereof that are generally non-toxic to the recipient and do not cause any significant adverse effects to the subject.

### Methods of Eradicating Cells in a Growth Arrest Phase

Methods of using functionalized nanoclusters to eradicate cells in a growth arrest phase are provided. The subject methods utilize nanoclusters functionalized with adenosine triphosphate (ATP) or an analogue thereof to kill dormant cells that have entered a non-growing or extremely slow-growing physiological state. Without being bound by theory, the nanoclusters deliver ATP (or an analogue thereof) to cells at a non-physiological concentration and disrupt cytoplasmic Mg²⁺ homeostasis by chelating Mg²⁺. In bacteria, increasing ATP in the cytoplasm to a non-physiological level results in chelation of Mg²⁺ and collapses the cytoplasmic pool of free Mg²⁺, which causes bacterial cell death during the growth arrest phase. A secondary mechanism may operate in bacteria involving inhibition of proteolytic activity. Degradation of misfolded and nonfunctional proteins by ATP-dependent proteases depends on the cytoplasmic ATP and Mg²⁺ concentration. For example, FtsH proteases are inner membrane ATP-dependent proteases that digest misassembled or damaged membrane proteins and short-lived water-soluble enzymes and transcription factors. ATP hydrolysis and exchange within the nucleotide pockets of FtsH are primarily responsible for inducing the conformational rearrangements required for substrate processing. High ATP conditions obstruct access to the FtsH binding pocket access by promoting a closed conformation and inhibiting proteolytic activity. Thus, delivery of ATP (or an analog thereof) to bacterial cells using the functionalized nanoclusters, described herein, may interfere with removal of misassembled or damaged proteins and contribute to cell death.

In particular, nanoclusters functionalized with ATP or an analogue thereof can be used for killing dormant cells in biofilms, which have entered a non-growing or extremely slow-growing physiological state, and as a result have become resistant to antimicrobial drugs. Such dormant cells in biofilms are referred to herein as “persister cells” because of their ability to persist after other active bacterial cells have been eradicated by the immune system and antimicrobial agents. Persister cells are often associated with chronic infections because of the difficulty of eradicating them with conventional antibiotic treatment. The methods described herein are especially useful for treating chronic infections to render persister cells in biofilms that have entered a growth arrest phase more susceptible to antibiotic treatment.

Nanoclusters functionalized with ATP (or an analogue thereof) can also be used to kill tumor cells. Without being bound by theory, the ATP-binding receptor, P2X7, responds to high levels of ATP by triggering a cascade of reactions that results in ATP-dependent cytotoxicity. Treatment of human tumor cells with ADP or ATP results in arrest of growth in the S phase of the cell cycle. Nanoclusters functionalized with ATP (or an analogue thereof) also induce endoplasmic reticulum (ER) stress in cancerous cells, which inhibits cancer growth. Therefore, ATP (or an analog thereof) can be delivered to tumor cells, using the functionalized nanoclusters described herein, to induce ATP-dependent tumor cytotoxicity, ER stress, and/or growth arrest of tumor cells.

In addition, nanoclusters functionalized with adenosine triphosphate (ATP) (or an analogue thereof) can be used to increase phagocytic clearance in a tissue, reduce NLRP3 activation, reduce IL-1beta-mediated inflammation, reduce microglial inflammation, and/or reduce oxidative stress in a subject.

In certain embodiments, a nanocluster is conjugated to a nucleotide such as, but not limited to, ATP or a phosphorothioate analog, a deoxyribonucleotide analog, a 7-deaza purine nucleotide analog, or a phosphomethylphosphonic acid adenylate ester thereof. Exemplary, phosphorothioate analogs include, without limitation, ATPαS, ATPβS, or ATPγS. Exemplary deoxyribonucleotide analogs include, without limitation, deoxyadenosine triphosphate (dATP). Exemplary 7-deaza purine nucleotide analogs include, without limitation, 7-deazaadenosine-5′-triphosphate (7-deaza-ATP). Exemplary phosphomethylphosphonic acid adenylate ester analogs include, without limitation, β,γ-methyleneadenosine 5′-triphosphate (AMP-PCP).

The nanocluster is typically spherical in shape, but nanoclusters having other shapes may also be used. For example, the nanocluster may have a shape such as, but not limited to, a sphere, a spheroid (e.g., an oblate or prolate spheroid), an ellipsoid, a rod, a cone, a cube, a cuboid (e.g., a hexahedron), a pyramid, an icosahedron, a truncated icosahedron, or an irregular shape, etc. In certain instances, combinations of different shapes of nanoclusters may be included in a composition. In some embodiments, the nanocluster is substantially spherical in shape, and thus may have dimensions measured as a diameter of a sphere. In certain embodiments, the nanocluster has a centered diameter distribution ranging from about 1 nm to about 10 nm, including any diameter within this range such as 0.5 nm, 0.75 nm, 1 nm, 1.25 nm, 1.5 nm, 1.75 nm, 2 nm, 2.25 nm, 2.5 nm, 2.75 nm, 3 nm, 3.25 nm, 3.5 nm, 3.75 nm, 4 nm, 4.5 nm, 5 nm, 5.5 nm, 6 nm, 6.5 nm, 7 nm, 7.5 nm, 8 nm, 8.5 nm, 9 nm, 9.5 nm, or 10 nm. In some instances, a substantially spherical nanocluster has an average diameter of 2 nm or less. In some embodiments, a substantially spherical nanocluster has an average diameter of about 1 nm to about 2 nm.

The nanocluster may comprise, for example, a metal, a ceramic, carbon-based nanomaterials, silicon or silica, boron, polymers, lipids, or proteins. In certain embodiments, the nanocluster comprises a metallic core conjugated to a nucleotide. The metallic core may comprise a single type of metal atom or more than one type of metal atom, such as two or three, or more different types of metal atoms. In some embodiments, the nanocluster comprises a metal including, without limitation, one or more of gold, silver, platinum, titanium, palladium, rhodium, ruthenium, tin, nickel, copper, aluminum, or an oxide, carbide, nitride, or alloy thereof. In other embodiments, the nanocluster is composed of an oxide of silicon, aluminum, a transition metal (e.g., titanium, zirconium, and the like), aluminosilicate, boron nitride, or a combination thereof. Exemplary materials that may be used in nanoclusters include, but are not limited to, silicon dioxide (e.g., silica), titanium dioxide, silicon-aluminum-oxide, aluminum oxide, and iron oxide. In some instances, the nanocluster is composed of other inorganic materials, such as, but not limited to, diatomaceous earth, calcium hydroxyapatite, and the like. Nanoclusters may also be composed of hydrophobic polymers such as, but not limited to, polylactide; polylactic acid; polyolefins, such as polyethylene, poly(isobutene), poly(isoprene), poly(4-methyl-1-pentene), polypropylene, ethylene-propylene copolymers, and ethylenepropylene-hexadiene copolymers; ethylene-vinyl acetate copolymers; and styrene polymers, such as poly(styrene), poly(2-methylstyrene), styrene-acrylonitrile copolymers, and styrene-2,2,3,3,-tetrafluoro-propyl methacrylate copolymers. Nanoclusters may also be composed of natural polymers such as proteins, including, without limitation, albumin, silk, keratin, collagen, elastin, corn zein, and soy protein-based nanoclusters; or polysaccharide-based polymers, including, without limitation, chitosan, hyaluronic acid, alginate, glucan, dextran, and cyclodextrin-based nanoclusters. Carbon-based nanoclusters may include, without limitation, carbon nanotubes, graphite, graphene, fullerenes and nanodiamonds. Combinations of the above materials may also be included in nanoclusters. In certain embodiments, the nanocluster is biocompatible with human cells.

In certain embodiments, the nanocluster is linked to an internalization sequence, a protein transduction domain, or a cell penetrating peptide to facilitate entry into a cell. Cell penetrating peptides that can be used include, but are not limited to, human immunodeficiency virus (HIV)-Tat, penetratin, transportan, octaarginine, nonaarginine, antennapedia, TP10, Buforin II, MAP (model amphipathic peptide), K-FGF, Ku70, mellittin, pVEC, Pep-1, SynB1, Pep-7, CADY, GALA, pHLIP, KALA, R7W, and HN-1, which can readily transport nanoclusters across plasma membranes (see, e.g., Lai et al. (2023) Bioconjug. Chem. 34(1):228-237; Peng et al. (2014) Biomaterials 35(21):5605-5618; Jones et al. (2012) J. Control Release 161(2):582-591; Fonseca et al. (2009) Adv. Drug Deliv. Rev. 61(11):953-64; Schwarze et al. (1999) Science. 285(5433):1569-72; Derossi et al. (1996) J. Biol. Chem. 271(30):18188-18193; Fuchs et al. (2004) Biochemistry 43(9):2438-2444; and Yuan et al. (2002) Cancer Res. 62(15):4186-4190).

### Conjugation

Surface functionalization of nanoclusters may be performed by any method known in the art. Functionalization of a nanocluster involves conjugation of a nucleotide (e.g., ATP, dATP, ATPαS, ATPβS, ATPγS, 7-deaza-ATP, or AMP-PCP) to a molecule on the outer surface of the nanocluster. A surface coating may be applied to nanoclusters to introduce functional groups to facilitate attachment of agents. For example, gold nanoclusters with surface coatings comprising thiol, carboxyl, amine, aldehyde, hydroxyl, or azide groups, polyethylene glycol (PEG), dextran, streptavidin, or maleimide and compounds to facilitate bioconjugation are commercially available from a number of companies (e.g., SigmaAldrich (St. Louis, MO), and Cytodiagnostics (Burlington, Ontario, Canada), Creative Diagnostics (Shirley, NY), and Nanocs (New York, NY)). An agent may be conjugated to a nanocluster directly or indirectly through a linker. Exemplary linkers include, without limitation, thioC6 linker (thiohexyl), PEG polymers, diethylenetriaminepentaacetic acid (DTPA), 1,4,7,10-tetraazacyclododecane-1,4,7,10-tetraacetic acid (DOTA), and hydrazide compounds. For a discussion of bioconjugation techniques, see, e.g., Chemistry of Bioconjugates: Synthesis, Characterization, and Biomedical Applications (R. Narain ed., Wiley, 2014), G. T. Hermanson Bioconjugate Techniques (Academic Press, 3^{rd} edition, 2013), and Bioconjugation Protocols: Strategies and Methods (Methods in Molecular Biology, S. S. Mark ed., Humana Press, 2^{nd} edition, 2011), Avvakumova et al. (2014) Trends Biotechnol. 32(1):11-20., Couto et al. (2017) Crit Rev Biotechnol. 37(2):238-250, Sivaram et al. (2018) Adv. Healthc Mater. 7 (1), van Vught et al. (2014) Comput Struct Biotechnol J. 9:e201402001; Massa et al. (2016) Expert Opin Drug Deliv 13:1-15; Yeh et al. (2015) PLoS One 10(7):e0129681; Freise et al. (2015) Mol Immunol. 67(2 Pt A):142-152; herein incorporated by reference in their entireties.

A variety of conjugation methods and chemistries can be used to conjugate nucleotides or other agents to a nanocluster. Various zero-length, homo-bifunctional, and hetero-bifunctional crosslinking reagents can be used. Zero-length crosslinking reagents include direct conjugation of two intrinsic chemical groups with no introduction of extrinsic material. Agents that catalyze formation of a disulfide bond belong to this category. Another example is reagents that induce condensation of a carboxyl and a primary amino group to form an amide bond such as carbodiimides, ethylchloroformate, Woodward's reagent K (2-ethyl-5-phenylisoxazolium-3′-sulfonate), and carbonyldiimidazole. Homo- and hetero-bifunctional reagents generally contain two identical or two non-identical sites, respectively, which may be reactive with amino, sulfhydryl, guanidino, indole, or nonspecific groups.

Suitable amino-reactive groups include, but are not limited to, N-hydroxysuccinimide (NHS) esters, imidoesters, isocyanates, acylhalides, arylazides, p-nitrophenyl esters, aldehydes, and sulfonyl chlorides. Suitable sulfhydryl-reactive groups include, but are not limited to, maleimides, alkyl halides, pyridyl disulfides, and thiophthalimides. In other embodiments, carbodiimides soluble in both water and organic solvent, are used as carboxyl-reactive reagents. These compounds react with free carboxyl groups forming a pseudourea that can then couple to available amines, yielding an amide linkage.

In some embodiments, a nucleotide or other agent is conjugated to a nanocluster using a homobifunctional crosslinker. In some embodiments, the homobifunctional crosslinker is reactive with primary amines. Homobifunctional crosslinkers that are reactive with primary amines include NHS esters, imidoesters, isothiocyanates, isocyanates, acylhalides, arylazides, p-nitrophenyl esters, aldehydes, and sulfonyl chlorides. Non-limiting examples of homobifunctional NHS esters include disuccinimidyl glutarate (DSG), disuccinimidyl suberate (DSS), bis(sulfosuccinimidyl)suberate (BS), disuccinimidyl tartarate (DST), disulfosuccinimidyl tartarate (sulfo-DST), bis-2-(succinimidooxycarbonyloxy)ethylsulfone (BSOCOES), bis-2-(sulfosuccinimidooxycarbonyloxy)ethylsulfone (sulfo-BSOCOES), ethylene glycolbis(succinimidylsuccinate) (EGS), ethylene glycolbis(sulfosuccinimidylsuccinate) (sulfo-EGS), dithiobis(succinimidylpropionate (DSP), and dithiobis(sulfosuccinimidylpropionate (sulfo-DSP). Non-limiting examples of homobifunctional imidoesters include dimethyl malonimidate (DMM), dimethyl succinimidate (DMSC), dimethyl adipimidate (DMA), dimethyl pimelimidate (DMP), dimethyl suberimidate (DMS), dimethyl-3,3′-oxydipropionimidate (DODP), dimethyl-3,3′-(methylenedioxy)dipropionimidate (DMDP), dimethyl-,3′-(dimethylenedioxy)dipropionimidate (DDDP), dimethyl-3,3′-(tetramethylenedioxy)dipropionimidate (DTDP), and dimethyl-3,3′-dithiobispropionimidate (DTBP).

Non-limiting examples of homobifunctional isothiocyanates include: p-phenylenediisothiocyanate (DITC), and 4,4′-diisothiocyano-2,2′-disulfonic acid stilbene (DIDS). Non-limiting examples of homobifunctional isocyanates include xylene-diisocyanate, toluene-2,4-diisocyanate, toluene-2-isocyanate-4-isothiocyanate, 3-methoxydiphenylmethane-4,4′-diisocyanate, 2,2′-dicarboxy-4,4′-azophenyldiisocyanate, and hexamethylenediisocyanate. Non-limiting examples of homobifunctional arylhalides include 1,5-difluoro-2,4-dinitrobenzene (DFDNB), and 4,4′-difluoro-3,3′-dinitrophenyl-sulfone. Non-limiting examples of homobifunctional aliphatic aldehyde reagents include glyoxal, malondialdehyde, and glutaraldehyde. Non-limiting examples of homobifunctional acylating reagents include nitrophenyl esters of dicarboxylic acids. Non-limiting examples of homobifunctional aromatic sulfonyl chlorides include phenol-2,4-disulfonyl chloride, and alpha-naphthol-2,4-disulfonyl chloride. Non-limiting examples of additional amino-reactive homobifunctional reagents include erythritolbiscarbonate, which reacts with amines to give biscarbamates.

In some embodiments, the homobifunctional crosslinker is reactive with free sulfhydryl groups. Homobifunctional crosslinkers reactive with free sulfhydryl groups include, e.g., maleimides, pyridyl disulfides, and alkyl halides. Non-limiting examples of homobifunctional maleimides include bismaleimidohexane (BMH), N,N′-(1,3-phenylene)bismaleimide, N,N′-(1,2-phenylene)bismaleimide, azophenyldimaleimide, and bis(N-maleimidomethyl)ether. Non-limiting examples of homobifunctional pyridyl disulfides include 1,4-di-3′-(2′-pyridyldithio)propionamidobutane (DPDPB). Non-limiting examples of homobifunctional alkyl halides include 2,2′-dicarboxy-4,4′-diiodoacetamidoazobenzene, α,α′-diiodo-p-xylenesulfonic acid, α,α′-dibromo-p-xylenesulfonic acid, N,N′-bis(b-bromoethyl)benzylamine, N,N′-di(bromoacetyfiphenylhydrazine, and 1,2-di(bromoacetyfiamino-3-phenylpropane.

In some embodiments, a nucleotide or other agent is conjugated to a nanocluster using a heterobifunctional reagent. Suitable heterobifunctional reagents include amino-reactive reagents comprising a pyridyl disulfide moiety; amino-reactive reagents comprising a maleimide moiety; amino-reactive reagents comprising an alkyl halide moiety; and amino-reactive reagents comprising an alkyl dihalide moiety.

Non-limiting examples of hetero-bifunctional reagents with a pyridyl disulfide moiety and an amino-reactive NHS ester include N-succinimidyl-3-(2-pyridyldithio)propionate (SPDP), succinimidyl 6-3-(2-pyridyldithio)propionamidohexanoate (LC-SPDP), sulfosuccinimidyl 6-3-(2-pyridyldithio)propionamidohexanoate (sulfo-LCSPDP), 4-succinimidyloxycarbonyl-α-methyl-α-(2-pyridyldithio)toluene (SMPT), and sulfosuccinimidyl 6-α-methyl-α-(2-pyridyldithio)toluamidohexanoate (sulfo-LC-SMPT).

Non-limiting examples of heterobifunctional reagents comprising a maleimide moiety and an amino-reactive NHS ester include succinimidyl maleimidylacetate (AMAS), succinimidyl 3-maleimidylpropionate (BMPS), N-gamma-maleimidobutyryloxysuccinimide ester (GMBS)N-gamma-maleimidobutyryloxysulfosuccinimide ester (sulfo-GMBS) succinimidyl 6-maleimidylhexanoate (EMCS), succinimidyl 3-maleimidylbenzoate (SMB), m-maleimidobenzoyl-N-hydroxysuccinimide ester (MBS), m-maleimidobenzoyl-N-hydroxysulfosuccinimide ester (sulfo-MBS), succinimidyl 4-(N-maleimidomethylicyclohexane-1-carboxylate (SMCC), sulfosuccinimidyl 4-(N-maleimidomethyl)cyclohexane-1-carboxylate (sulfo-SMCC), succinimidyl 4-(p-maleimidophenyl)butyrate (SMPB), and sulfosuccinimidyl 4-(p-maleimidophenyl)butyrate (sulfo-SMPB).

Non-limiting examples of heterobifunctional reagents comprising an alkyl halide moiety and an amino-reactive NHS ester include N-succinimidyl-(4-iodoacetyl)aminobenzoate (SIAB), sulfosuccinimidyl-(4-iodoacetyl)aminobenzoate (sulfo-SIAB), succinimidyl-6-(iodoacetyl)aminohexanoate (SIAX), succinimidyl-6-(6-((iodoacetyl)-amino)hexanoylamino)hexanoate (SIAXX), succinimidyl-6-(((4-(iodoacetyl)-amino)methyl)-cyclohexane-1-carbonyl)ami-nohexanoate (SIACX), and succinimidyl-4((iodoacetyl)-amino)methylcyclohexane-1-carboxylate (SIAC).

A non-limiting example of a hetero-bifunctional reagent comprising an amino-reactive NHS ester and an alkyl dihalide moiety is N-hydroxysuccinimidyl 2,3-dibromopropionate (SDBP). A non-limiting example of a hetero-bifunctional reagent comprising an alkyl halide moiety and an amino-reactive p-nitrophenyl ester moiety includes p-nitrophenyl iodoacetate (NPIA).

In another example, a 3-ThioC6 linker can be used to functionalize a nucleotide or other agent with a thiol group to facilitate attachment to nanoclusters. For example, the 3-ThioC6 linker can be used to add a thiol group to a nucleotide. The free thiol can be used as a reactive functional group to attach maleimide compounds or for conjugation through disulfide linkages.

An alternative bioconjugation method uses click chemistry. Click chemistry reactions include the Huisgen 1,3-dipolar cycloaddition copper catalyzed reaction (Tornoe et al., 2002, J Organic Chem 67:3057-64), cycloaddition reactions such as Diels-Alder reactions, nucleophilic substitution reactions (especially to small strained rings like epoxy and aziridine compounds), reactions involving formation of urea compounds, and reactions involving carbon-carbon double bonds, such as alkynes in thiol-yne reactions. See, e.g., Kolb et al., 2004, Angew Chem Int Ed 40:3004-31; Evans, 2007, Aust J Chem 60:384-95; Millward et al. (2013) Integr Biol (Camb) 5(1):87-95), Lallana et al. (2012) Pharm Res 29(1):1-34, Gregoritza et al. (2015) Eur J Pharm Biopharm. 97(Pt B):438-453, Musumeci et al. (2015) Curr Med Chem. 22(17):2022-2050, Mckay et al. (2014) Chem Biol21(9):1075-1101, Ulrich et al. (2014) Chemistry 20(1):34-41, Pasini (2013) Molecules 18(8):9512-9530, and Wangler et al. (2010) Curr Med Chem. 17(11):1092-1116; herein incorporated by reference in their entireties.

### Pharmaceutical Compositions

A functionalized nanocluster conjugated to a nucleotide (e.g., ATP, dATP, ATPαS, ATPβS, ATPγS, 7-deaza-ATP, or AMP-PCP), as described herein, can be formulated into a pharmaceutical composition optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition can also include an antimicrobial agent for preventing or deterring microbial growth. Nonlimiting examples of antimicrobial agents include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the nanoclusters or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, New Jersey); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the nanoclusters (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, NJ (1998), and Kibbe, A. H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

The pharmaceutical preparations herein can also be housed in a syringe, an implantation device, or the like, depending upon the intended mode of delivery and use. Preferably, the compositions comprising nanoclusters are in unit dosage form, meaning an amount of a composition appropriate for a single dose, in a premeasured or pre-packaged form.

The compositions herein may optionally include one or more additional agents, such as antibiotics, adjuvants, immunostimulatory agents, vaccines, and/or other medications used to treat a subject for an infection. Compounded preparations may include nanoclusters and one or more other agents for treating an infection, such as, but not limited to, antibiotics including broad spectrum, bactericidal, or bacteriostatic antibiotics such as penicillins including penicillin G, penicillin V, procaine penicillin, benzathine penicillin, veetids (Pen-Vee-K), piperacillin, pipracil, pfizerpen, temocillin, negaban, ticarcillin, and Ticar; penicillin combinations such as amoxicillin/clavulanate, augmentin, ampicillin/sulbactam, unasyn, piperacillin/tazobactam, zosyn, ticarcillin/clavulanate, and timentin; tetacyclines such as chlortetracycline, doxycycline, demeclocycline, eravacycline, lymecycline, meclocycline, methacycline, minocycline, omadacycline, oxytetracycline, rolitetracycline, sarecycline, tetracycline, and tigecycline; cephalosporins such as cefacetrile (cephacetrile), cefadroxil (cefadroxyl; duricef), cefalexin (cephalexin; keflex), cefaloglycin (cephaloglycin), cefalonium (cephalonium), cefaloridine (cephaloradine), cefalotin (cephalothin; keflin), cefapirin (cephapirin; cefadryl), cefatrizine, cefazaflur, cefazedone, cefazolin (cephazolin; ancef, kefzol), cefradine (cephradine; velosef), cefroxadine, ceftezole, cefaclor (ceclor, distaclor, keflor, raniclor), cefonicid (monocid), cefprozil (cefproxil; cefzil), cefuroxime (zefu, zinnat, zinacef, ceftin, biofuroksym, xorimax), cefuzonam, loracarbef (lorabid) cefbuperazone, cefmetazole (zefazone), cefminox, cefotetan (cefotan), cefoxitin (mefoxin), cefotiam (pansporin), cefcapene, cefdaloxime, cefdinir (sefdin, zinir, omnicef, kefnir), cefditoren, cefetamet, cefixime (fixx, zifi, suprax), cefmenoxime, cefodizime, cefotaxime (claforan), cefovecin (convenia), cefpimizole, cefpodoxime (vantin, pecef, simplicef), cefteram, ceftamere (enshort), ceftibuten (cedax), ceftiofur (naxcel, excenel), ceftiolene, ceftizoxime (cefizox), ceftriaxone (rocephin), cefoperazone (cefobid), ceftazidime (meezat, fortum, fortaz), latamoxef (moxalactam), cefclidine, cefepime (maxipime), cefluprenam, cefoselis, cefozopran, cefpirome (cefrom), cefquinome, flomoxef, ceftobiprole, ceftaroline, ceftolozane, cefaloram, cefaparole, cefcanel, cefedrolor, cefempidone, cefetrizole, cefivitril, cefmatilen, cefmepidium, cefoxazole, cefrotil, cefsumide, cefuracetime, ceftioxide, and nitrocefin; quinolones//fluoroquinolones such as flumequine (Flubactin), oxolinic acid (Uroxin), rosoxacin (Eradacil), cinoxacin (Cinobac), nalidixic acid (NegGam, Wintomylon), piromidic acid (Panacid), pipemidic acid (Dolcol), ciprofloxacin (Zoxan, Ciprobay, Cipro, Ciproxin), fleroxacin (Megalone, Roquinol), lomefloxacin (Maxaquin), nadifloxacin (Acuatim, Nadoxin, Nadixa), norfloxacin (Lexinor, Noroxin, Quinabic, Janacin), ofloxacin (Floxin, Oxaldin, Tarivid), pefloxacin (Peflacine), rufloxacin (Uroflox), enoxacin (Enroxil, Penetrex), balofloxacin (Baloxin), grepafloxacin (Raxar), levofloxacin (Cravit, Levaquin), pazufloxacin (Pasil, Pazucross), sparfloxacin (Zagam), temafloxacin (Omniflox), tosufloxacin (Ozex, Tosacin), clinafloxacin, gatifloxacin (Zigat, Tequin, Zymar-ophthalmic), moxifloxacin (Avelox, Vigamox), sitafloxacin (Gracevit), prulifloxacin (Quisnon), besifloxacin (Besivance), delafloxacin (Baxdela), gemifloxacin (Factive) and trovafloxacin (Trovan), ozenoxacin, danofloxacin (Advocin, Advocid), difloxacin (Dicural, Vetequinon), enrofloxacin (Baytril), ibafloxacin (Ibaflin), marbofloxacin (Marbocyl, Zenequin), orbifloxacin (Orbax, Victas), and sarafloxacin (Floxasol, Saraflox, Sarafin); macrolides such as azithromycin, clarithromycin, erythromycin, fidaxomicin, telithromycin, carbomycin A, josamycin, kitasamycin, midecamycin/midecamycin acetate, oleandomycin, solithromycin, spiramycin, troleandomycin, tylosin/tylocine, roxithromycin, telithromycin, cethromycin, solithromycin, tacrolimus, pimecrolimus, sirolimus, amphotericin B, nystatin, and cruentaren; sulfonamides such as sulfonamide, sulfacetamide, sulfadiazine, sulfadimidine, sulfafurazole (sulfisoxazole), sulfisomidine (sulfaisodimidine), sulfamethoxazole, sulfamoxole, sulfanitran, sulfadimethoxine, sulfamethoxypyridazine, sulfametoxydiazine, sulfadoxine, sulfametopyrazine, and terephtyl; aminoglycosides such as kanamycin A, amikacin, tobramycin, dibekacin, gentamicin, sisomicin, netilmicin, neomycins B, C, neomycin E (paromomycin), streptomycin, plazomicin, amikin, garamycin, kantrex, neo-fradin, netromycin, nebcin, humatin, spectinomycin (Bs), and trobicin; carbapenems such as imipenem, meropenem, ertapenem, doripenem, panipenem/betamipron, biapenem, tebipenem, razupenem (PZ-601), lenapenem, tomopenem, and thienamycin (thienpenem); ansamycins such as geldanamycin, herbimycin, rifaximin, and xifaxan; carbacephems such as loracarbef and lorabid; carbapenems such as ertapenem, invanz, doripenem, doribax, imipenem/cilastatin, primaxin, meropenem, and merrem; glycopeptides such as teicoplanin, targocid, vancomycin, vancocin, telavancin, vibativ, dalbavancin, dalvance, oritavancin, and orbactiv; lincosamides such as clindamycin, cleocin, lincomycin, and lincocin; lipopeptides such as daptomycin and cubicin; macrolides such as azithromycin, zithromax, sumamed, xithrone, clarithromycin, biaxin, dirithromycin, dynabac, erythromycin, erythocin, erythroped, roxithromycin, troleandomycin, tao, telithromycin, ketek, spiramycin, and rovamycine; monobactams such as aztreonam and azactam; nitrofurans such as furazolidone, furoxone, nitrofurantoin, macrodantin, and macrobid; oxazolidinones such as linezolid, zyvox, vrsa, posizolid, radezolid, and torezolid; polypeptides such as bacitracin, colistin, coly-mycin-S, and polymyxin B; drugs against mycobacteria such as clofazimine, lamprene, dapsone, avlosulfon, capreomycin, capastat, cycloserine, seromycin, ethambutol, myambutol, ethionamide, trecator, isoniazid, I.N.H., pyrazinamide, aldinamide, rifampicin, rifadin, rimactane, rifabutin, mycobutin, rifapentine, priftin, and streptomycin; and other antibiotics such as arsphenamine, salvarsan, chloramphenicol, chloromycetin, fosfomycin, monurol, monuril, fusidic acid, fucidin, metronidazole, flagyl, mupirocin, bactroban, platensimycin, quinupristin/dalfopristin, synercid, thiamphenicol, tigecycline, tigacyl, tinidazole, tindamax fasigyn, trimethoprim, proloprim, and trimpex; adjuvants, including aluminum salts (alum), such as aluminum hydroxide, aluminum phosphate, aluminum sulfate, etc.; oil-in-water emulsion formulations; (saponin adjuvants; Complete Freund's Adjuvant (CFA) and Incomplete Freund's Adjuvant (IFA); cytokines, such as interleukins (IL-1, IL-2, IL-4, IL-5, IL-6, IL-7, IL-12, interferons, macrophage colony stimulating factor (M-CSF), tumor necrosis factor (TNF), etc.; detoxified mutants of a bacterial ADP-ribosylating toxin such as a cholera toxin (CT), pertussis toxin (PT), or an E. coli heat-labile toxin (LT); oligonucleotides comprising CpG motifs; as well as other immunostimulatory molecules; and vaccines against bacteria and infectious diseases, including any vaccine comprising bacterial antigenic proteins or attenuated or dead bacteria and, optionally, adjuvants for boosting an immune response against bacteria, such as vaccines against tuberculosis, diphtheria, tetanus, pertussis, Haemophilus influenzae type B, cholera, typhoid, Streptococcus pneumoniae, and the like.

In other embodiments, the compositions herein optionally include one or more additional agents for treating a subject for a fungal infection. Compounded preparations may include nanoclusters and one or more antifungal agents such as, but not limited to, amphotericin B, voriconazole, caspofungin, and fluconazole.

In yet other embodiments, the compositions herein optionally include one or more additional agents for treating a subject for a benign or malignant tumor, such as chemotherapeutic agents, targeted therapeutic agents, immunotherapeutic agents, radioisotopes, radiosensitizing drugs, and/or other medications. Compounded preparations may include nanoclusters and one or more other agents for treating tumors such as, but not limited to, chemotherapeutic agents such as abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon; targeted therapeutic agents such as tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade); Janus kinase inhibitors, such as tofacitinib; ALK inhibitors, such as crizotinib; Bcl-2 inhibitors, such as obatoclax and gossypol; PARP inhibitors, such as Iniparib and Olaparib; PI3K inhibitors, such as perifosine; VEGF receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar); and monoclonal antibodies, such as Rituximab (marketed as MabThera or Rituxan), Trastuzumab (Herceptin), Alemtuzumab, Cetuximab (marketed as Erbitux), Panitumumab, Bevacizumab (marketed as Avastin), and Ipilimumab (Yervoy); immunotherapeutic agents such as a cancer vaccine (e.g., E75 HER2-derived peptide vaccine, nelipepimut-S (NeuVax), Sipuleucel-T), antibody therapy (e.g., Trastuzumab, Ado-trastuzumab emtansine, Alemtuzumab, Ipilimumab, Ofatumumab, Nivolumab, Pembrolizumab, or Rituximab), cytokine therapy (e.g., interferons, including type I (IFNα and IFNβ), type II (IFNγ) and type III (IFNλ) and interleukins, including interleukin-2 (IL-2)), adjuvant immunochemotherapy (e.g., polysaccharide-K), adoptive T-cell therapy, and immune checkpoint blockade therapy; radioisotopes such as iodine-131, strontium-89, samarium-153, and radium-223; and radiosensitizing drugs such as Cisplatin, Nimorazole, and Cetuximab.

Alternatively, such agents can be contained in a separate composition from the composition comprising the nanoclusters and co-administered concurrently, before, or after the composition comprising the nanoclusters.

### Administration

In some embodiments, at least one therapeutically effective cycle of treatment with a composition comprising a functionalized nanocluster conjugated to a nucleotide (e.g., ATP, dATP, ATPαS, ATPβS, ATPγS, 7-deaza-ATP, or AMP-PCP), as described herein, will be administered to a subject to eradicate a pathogenic cell in a growth arrest phase. The pathogenic cell may be a prokaryotic cell or a eukaryotic cell. In some embodiments, the pathogenic cell is a bacterial cell, a fungal cell, or a human cell in a growth arrest phase.

In some embodiments, at least one therapeutically effective cycle of treatment with a composition comprising a functionalized nanocluster conjugated to a nucleotide (e.g., ATP, dATP, ATPαS, ATPβS, ATPγS, 7-deaza-ATP, or AMP-PCP), as described herein, is administered to a subject for treatment of a bacterial infection. Bacterial infections that can be treated by the methods described herein include bacterial infections caused by Gram negative bacteria such as, but not limited to, Acinetobacter (e.g., Acinetobacter baumannii), Actinobacillus, Bordetella, Brucella, Campylobacter, Cyanobacteria, Enterobacter (e.g., Enterobacter cloacae), Erwinia, Escherichia coli, Franciscella, Helicobacter (Helicobacter pylori), Hemophilus (e.g., Hemophilus influenzae), Klebsiella (e.g., Klebsiella pneumoniae), Legionella (e.g., Legionella pneumophila), Moraxella (e.g., Moraxella catarrhalis), Neisseria (e.g., Neisseria gonorrhoeae, Neisseria meningitidis), Pasteurella, Proteus (e.g., Proteus mirabilis), Pseudomonas (e.g., Pseudomonas aeruginosa), Salmonella (e.g., Salmonella enteritidis, Salmonella typhi), Serratia (e.g., Serratia marcescens), Shigella, Treponema, Vibrio (e.g., Vibrio cholerae), and Yersinia (e.g., Yersinia pestis), as well as Gram positive bacteria such as, but not limited to, Actinobacteria, such as Actinomyces (e.g., Actinomyces israelii), Arthrobacter, Bifidobacterium, Corynebacterium (e.g., Corynebacterium diphtheriae), Frankia, Micrococcus, Micromonospora, Mycobacterium (e.g., Mycobacterium tuberculosis, Mycobacterium leprae), Nocardia, Propionibacterium, and Streptomyces; Firmicutes, such as Bacilli, order Bacillales including Bacillus, Listeria (e.g., Listeria monocytogenes), and Staphylococcus (e.g., Staphylococcus aureus, Staphylococcus epidermidis), Bacilli (e.g., Bacilli anthracis, Bacilli cereus), order Lactobacilluses, including Enterococcus, Lactobacillus, Lactococcus, Leuconostoc, Pediococcus, and Streptococcus (e.g., Streptococcus pneumoniae, Streptococcus mutans, Streptococcus sanguinis, Streptococcus pyogenes), Clostridia (e.g., Clostridioides difficile, Clostridium perfringens, Clostridium botulinum, Clostridium tetani, Clostridium sordellii), including Acetobacterium, Clostridium, Eubacterium, Heliobacterium, Heliospirillum, Megasphaera, Pectinatus, Selenomonas, Zymophilus, and Sporomusa, Mollicutes, including Mycoplasma (e.g., Mycoplasma pneumoniae), Spiroplasma, Ureaplasma, and Erysipelothrix. By “therapeutically effective dose or amount” of a nanocluster conjugated to a nucleotide (e.g., ATP, dATP, ATPαS, ATPβS, ATPγS, 7-deaza-ATP, or AMP-PCP) for treatment of a bacterial infection, wherein the bacteria are in a growth arrest phase, is intended an amount that, when administered alone or in combination with an antibiotic, as described herein, brings about a positive therapeutic response, such as improved recovery from an infection, including any infection caused by Gram-positive or Gram-negative bacteria. In particular, a therapeutically effective dose or amount may eradicate persister cells as well as other bacterial cells, including planktonic bacteria and bacteria in biofilms that are in a growth arrest phase. The exact amount required will vary from subject to subject, depending on the species, age, and general condition of the subject, the severity of the condition being treated, the particular type of nanoclusters and their functionalization, other antimicrobial agents or drugs employed in combination, the mode of administration, and the like. An appropriate “effective” amount in any individual case may be determined by one of ordinary skill in the art using routine experimentation, based upon the information provided herein.

In some embodiments, at least one therapeutically effective cycle of treatment with a composition comprising a functionalized nanocluster conjugated to a nucleotide (e.g., ATP, dATP, ATPαS, ATPβS, ATPγS, 7-deaza-ATP, or AMP-PCP), as described herein, is administered to a subject for treatment of a fungal infection. By “therapeutically effective dose or amount” of a nanocluster conjugated to a nucleotide (e.g., ATP, dATP, ATPαS, ATPβS, ATPγS, 7-deaza-ATP, or AMP-PCP) for treatment of a fungal infection, wherein the fungi are in a growth arrest phase, is intended an amount that, when administered alone or in combination with an antifungal agent, as described herein, brings about a positive therapeutic response, such as improved recovery from a fungal infection.

In other embodiments, at least one therapeutically effective cycle of treatment with a composition comprising a functionalized nanocluster conjugated to a nucleotide (e.g., ATP, dATP, ATPαS, ATPβS, ATPγS, 7-deaza-ATP, or AMP-PCP), as described herein, will be administered to a subject for treatment of a tumor (e.g., a benign or malignant tumor). Such treatment induces ER stress, reduces cell growth and proliferation, and kills tumor cells. By “therapeutically effective dose or amount” of a nanocluster conjugated to a nucleotide (e.g., ATP, dATP, ATPαS, ATPβS, ATPγS, 7-deaza-ATP, or AMP-PCP) for treatment of a tumor is intended an amount that, when administered alone or in combination with an anti-tumor agent, as described herein, has anti-tumor activity. Therefore, for example, a positive therapeutic response would refer to one or more of the following improvements in the disease: (1) reduction in tumor size; (2) reduction in the number of cancer cells; (3) inhibition (i.e., slowing to some extent, preferably halting) of tumor growth; (4) inhibition (i.e., slowing to some extent, preferably halting) of cancer cell infiltration into peripheral organs; (5) inhibition (i.e., slowing to some extent, preferably halting) of tumor metastasis; and (6) some extent of relief from one or more symptoms associated with the cancer.

Such therapeutic responses may be further characterized as to degree of improvement. Thus, for example, an improvement may be characterized as a complete response. By “complete response” is documentation of the disappearance of all symptoms and signs of all measurable or evaluable disease confirmed by physical examination, laboratory, nuclear and radiographic studies (i.e., CT (computer tomography) and/or MRI (magnetic resonance imaging)), and other non-invasive procedures repeated for all initial abnormalities or sites positive at the time of entry into the study. Alternatively, an improvement in the disease may be categorized as being a partial response. By “partial response” is intended a reduction of greater than 50% in the sum of the products of the perpendicular diameters of all measurable lesions when compared with pretreatment measurements (for patients with evaluable response only, partial response does not apply).

In some embodiments, at least one therapeutically effective cycle of treatment with a composition comprising ATP is administered in combination with a composition comprising a functionalized nanocluster conjugated to a nucleotide (e.g., ATP, dATP, ATPαS, ATPβS, ATPγS, 7-deaza-ATP, or AMP-PCP) to the subject. The ATP can be administered prior to, concurrent with, or subsequent to the functionalized nanocluster. If provided at the same time as the functionalized nanocluster, the ATP can be provided in the same or in a different composition. Thus, the two agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering at least one therapeutically effective dose of a pharmaceutical composition comprising ATP and at least one therapeutically effective dose of a pharmaceutical composition comprising a functionalized nanocluster conjugated to a nucleotide (e.g., ATP, dATP, ATPαS, ATPβS, ATPγS, 7-deaza-ATP, or AMP-PCP) according to a particular dosing regimen. Administration of the separate pharmaceutical compositions can be at the same time (i.e., simultaneously) or at different times (i.e., sequentially, in either order, on the same day, or on different days), so long as the therapeutic effect of the combination of these substances is caused in the subject undergoing therapy.

The compositions comprising nanoclusters and/or ATP are typically, although not necessarily, administered orally, via injection (subcutaneously, intravenously, or intramuscularly), by infusion, topically, or locally. Additional modes of administration are also contemplated, such as intra-arterial, intravascular, pulmonary, intralesional, intraparenchymatous, rectal, transdermal, transmucosal, intrathecal, intraocular, intraperitoneal, intratumoral, and so forth.

The preparations according to the invention are also suitable for local treatment. For example, compositions comprising nanoclusters and/or ATP may be administered directly to the site of infected tissue or the site of a tumor. For example, the particular preparation and appropriate method of administration can be chosen to target the nanoclusters and/or ATP to sites of chronic infection, sites of bacterial biofilms where persister cells typically reside and require eradication, or locally to benign or malignant tumors.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising nanoclusters and/or other agents may be administered using the same or different routes of administration in accordance with any medically acceptable method known in the art.

In another embodiment, the pharmaceutical compositions comprising nanoclusters and/or ATP, and/or other agents are administered prophylactically, e.g., to prevent infection. Such prophylactic uses will be of particular value for subjects who are immunodeficient, patients who have been treated with immunosuppressive agents, or who have a genetic predisposition or disease (e.g., acquired immunodeficiency syndrome (AIDS), cancer, diabetes, or cystic fibrosis) that makes them prone to developing infections or tumors, or subjects in an environment where exposure to infectious bacteria or fungi or carcinogens is likely.

In another embodiment, the pharmaceutical compositions comprising nanoclusters and/or ATP, and/or antibiotics, antifungal agents, or antitumor agents, and/or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

Those of ordinary skill in the art will appreciate which conditions the nanoclusters can effectively treat. The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case.

In certain embodiments, multiple therapeutically effective doses of a composition comprising nanoclusters and/or ATP and/or other agents will be administered according to a daily dosing regimen or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, once a week, every other week, and so forth. For example, in some embodiments, a composition comprising nanoclusters will be administered once-weekly, twice-weekly or thrice-weekly for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., once-weekly, twice-weekly or thrice-weekly administration of a therapeutically effective dose) for one or more weekly cycles until the desired therapeutic response is achieved. The agents can be administered by any acceptable route of administration as noted herein below. The amount administered will depend on the potency of the nanocluster and its type of functionalization, the magnitude of the effect desired, and the route of administration.

Nanoclusters (again, preferably provided as part of a pharmaceutical preparation) can be administered alone or in combination with ATP and/or one or more other therapeutic agents such as other agents for treating an infection, including, but not limited to, antibiotics including broad spectrum, bactericidal, or bacteriostatic antibiotics such as penicillins including penicillin G, penicillin V, procaine penicillin, benzathine penicillin, veetids (Pen-Vee-K), piperacillin, pipracil, pfizerpen, temocillin, negaban, ticarcillin, and Ticar; penicillin combinations such as amoxicillin/clavulanate, augmentin, ampicilin/sulbactam, unasyn, piperacillin/tazobactam, zosyn, ticarcillin/clavulanate, and timentin; tetacyclines such as chlortetracycline, doxycycline, demeclocycline, eravacycline, lymecycline, meclocycline, methacycline, minocycline, omadacycline, oxytetracycline, rolitetracycline, sarecycline, tetracycline, and tigecycline; cephalosporins such as cefacetrile (cephacetrile), cefadroxil (cefadroxyl; duricef), cefalexin (cephalexin; keflex), cefaloglycin (cephaloglycin), cefalonium (cephalonium), cefaloridine (cephaloradine), cefalotin (cephalothin; keflin), cefapirin (cephapirin; cefadryl), cefatrizine, cefazaflur, cefazedone, cefazolin (cephazolin; ancef, kefzol), cefradine (cephradine; velosef), cefroxadine, ceftezole, cefaclor (ceclor, distaclor, keflor, raniclor), cefonicid (monocid), cefprozil (cefproxil; cefzil), cefuroxime (zefu, zinnat, zinacef, ceftin, biofuroksym, xorimax), cefuzonam, loracarbef (lorabid) cefbuperazone, cefmetazole (zefazone), cefminox, cefotetan (cefotan), cefoxitin (mefoxin), cefotiam (pansporin), cefcapene, cefdaloxime, cefdinir (sefdin, zinir, omnicef, kefnir), cefditoren, cefetamet, cefixime (fixx, zifi, suprax), cefmenoxime, cefodizime, cefotaxime (claforan), cefovecin (convenia), cefpimizole, cefpodoxime (vantin, pecef, simplicef), cefteram, ceftamere (enshort), ceftibuten (cedax), ceftiofur (naxcel, excenel), ceftiolene, ceftizoxime (cefizox), ceftriaxone (rocephin), cefoperazone (cefobid), ceftazidime (meezat, fortum, fortaz), latamoxef (moxalactam), cefclidine, cefepime (maxipime), cefluprenam, cefoselis, cefozopran, cefpirome (cefrom), cefquinome, flomoxef, ceftobiprole, ceftaroline, ceftolozane, cefaloram, cefaparole, cefcanel, cefedrolor, cefempidone, cefetrizole, cefivitril, cefmatilen, cefmepidium, cefoxazole, cefrotil, cefsumide, ceftioxide, cefuracetime, and nitrocefin; quinolones//fluoroquinolones such as flumequine (Flubactin), oxolinic acid (Uroxin), rosoxacin (Eradacil), cinoxacin (Cinobac), nalidixic acid (NegGam, Wintomylon), piromidic acid (Panacid), pipemidic acid (Dolcol), ciprofloxacin (Zoxan, Ciprobay, Cipro, Ciproxin), fleroxacin (Megalone, Roquinol), lomefloxacin (Maxaquin), nadifloxacin (Acuatim, Nadoxin, Nadixa), norfloxacin (Lexinor, Noroxin, Quinabic, Janacin), ofloxacin (Floxin, Oxaldin, Tarivid), pefloxacin (Peflacine), rufloxacin (Uroflox), enoxacin (Enroxil, Penetrex), balofloxacin (Baloxin), grepafloxacin (Raxar), levofloxacin (Cravit, Levaquin), pazufloxacin (Pasil, Pazucross), sparfloxacin (Zagam), temafloxacin (Omniflox), tosufloxacin (Ozex, Tosacin), clinafloxacin, gatifloxacin (Zigat, Tequin, Zymar-ophthalmic), moxifloxacin (Avelox, Vigamox), sitafloxacin (Gracevit), prulifloxacin (Quisnon), besifloxacin (Besivance), delafloxacin (Baxdela), gemifloxacin (Factive) and trovafloxacin (Trovan), ozenoxacin, danofloxacin (Advocin, Advocid), difloxacin (Dicural, Vetequinon), enrofloxacin (Baytril), ibafloxacin (Ibaflin), marbofloxacin (Marbocyl, Zenequin), orbifloxacin (Orbax, Victas), and sarafloxacin (Floxasol, Saraflox, Sarafin); macrolides such as azithromycin, clarithromycin, erythromycin, fidaxomicin, telithromycin, carbomycin A, josamycin, kitasamycin, midecamycin/midecamycin acetate, oleandomycin, solithromycin, spiramycin, troleandomycin, tylosin/tylocine, roxithromycin, telithromycin, cethromycin, solithromycin, tacrolimus, pimecrolimus, sirolimus, amphotericin B, nystatin, and cruentaren; sulfonamides such as sulfonamide, sulfacetamide, sulfadiazine, sulfadimidine, sulfafurazole (sulfisoxazole), sulfisomidine (sulfaisodimidine), sulfamethoxazole, sulfamoxole, sulfanitran, sulfadimethoxine, sulfamethoxypyridazine, sulfametoxydiazine, sulfadoxine, sulfametopyrazine, and terephtyl; aminoglycosides such as kanamycin A, amikacin, tobramycin, dibekacin, gentamicin, sisomicin, netilmicin, neomycins B, C, neomycin E (paromomycin), streptomycin, plazomicin, amikin, garamycin, kantrex, neo-fradin, netromycin, nebcin, humatin, spectinomycin (Bs), and trobicin; carbapenems such as imipenem, meropenem, ertapenem, doripenem, panipenem/betamipron, biapenem, tebipenem, razupenem (PZ-601), lenapenem, tomopenem, and thienamycin (thienpenem); ansamycins such as geldanamycin, herbimycin, rifaximin, and xifaxan; carbacephems such as loracarbef and lorabid; carbapenems such as ertapenem, invanz, doripenem, doribax, imipenem/cilastatin, primaxin, meropenem, and merrem; glycopeptides such as teicoplanin, targocid, vancomycin, vancocin, telavancin, vibativ, dalbavancin, dalvance, oritavancin, and orbactiv; lincosamides such as clindamycin, cleocin, lincomycin, and lincocin; lipopeptides such as daptomycin and cubicin; macrolides such as azithromycin, zithromax, sumamed, xithrone, clarithromycin, biaxin, dirithromycin, dynabac, erythromycin, erythocin, erythroped, roxithromycin, troleandomycin, tao, telithromycin, ketek, spiramycin, and rovamycine; monobactams such as aztreonam and azactam; nitrofurans such as furazolidone, furoxone, nitrofurantoin, macrodantin, and macrobid; oxazolidinones such as linezolid, zyvox, vrsa, posizolid, radezolid, and torezolid; polypeptides such as bacitracin, colistin, coly-mycin-S, and polymyxin B; drugs against mycobacteria such as clofazimine, lamprene, dapsone, avlosulfon, capreomycin, capastat, cycloserine, seromycin, ethambutol, myambutol, ethionamide, trecator, isoniazid, I.N.H., pyrazinamide, aldinamide, rifampicin, rifadin, rimactane, rifabutin, mycobutin, rifapentine, priftin, and streptomycin; and other antibiotics such as arsphenamine, salvarsan, chloramphenicol, chloromycetin, fosfomycin, monurol, monuril, fusidic acid, fucidin, metronidazole, flagyl, mupirocin, bactroban, platensimycin, quinupristin/dalfopristin, synercid, thiamphenicol, tigecycline, tigacyl, tinidazole, tindamax fasigyn, trimethoprim, proloprim, and trimpex; adjuvants, including aluminum salts (alum), such as aluminum hydroxide, aluminum phosphate, aluminum sulfate, etc.; oil-in-water emulsion formulations; (saponin adjuvants; Complete Freund's Adjuvant (CFA) and Incomplete Freund's Adjuvant (IFA); cytokines, such as interleukins (IL-1, IL-2, IL-4, IL-5, IL-6, IL-7, IL-12, interferons, macrophage colony stimulating factor (M-CSF), tumor necrosis factor (TNF), etc.; detoxified mutants of a bacterial ADP-ribosylating toxin such as a cholera toxin (CT), pertussis toxin (PT), or an E. coli heat-labile toxin (LT); oligonucleotides comprising CpG motifs; as well as other immunostimulatory molecules; and vaccines such as vaccines against tuberculosis, diphtheria, tetanus, pertussis, Haemophilus influenzae type B, cholera, typhoid, and Streptococcus pneumoniae, and other vaccines comprising bacterial antigenic proteins or attenuated or dead bacteria for boosting an immune response against bacteria; antifungal agents such as amphotericin B, voriconazole, caspofungin, and fluconazole; or one or more other agents for treating tumors such as, but not limited to, chemotherapeutic agents such as abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon; targeted therapeutic agents such as tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade); Janus kinase inhibitors, such as tofacitinib; ALK inhibitors, such as crizotinib; Bcl-2 inhibitors, such as obatoclax and gossypol; PARP inhibitors, such as Iniparib and Olaparib; PI3K inhibitors, such as perifosine; VEGF receptor 2inhibitors, such as Apatinib; AN-152 (AEZS-108) doxorubicin linked to [D-Lys (6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar); and monoclonal antibodies, such as Rituximab (marketed as MabThera or Rituxan), Trastuzumab (Herceptin), Alemtuzumab, Cetuximab (marketed as Erbitux), Panitumumab, Bevacizumab (marketed as Avastin), and Ipilimumab (Yervoy); immunotherapeutic agents such as a cancer vaccine (e.g., E75 HER2-derived peptide vaccine, nelipepimut-S (NeuVax), Sipuleucel-T), antibody therapy (e.g., Trastuzumab, Ado-trastuzumab emtansine, Alemtuzumab, Ipilimumab, Ofatumumab, Nivolumab, Pembrolizumab, or Rituximab), cytokine therapy (e.g., interferons, including type I (IFNα and IFNβ), type II (IFNγ) and type III (IFNλ) and interleukins, including interleukin-2 (IL-2)), adjuvant immunochemotherapy (e.g., polysaccharide-K), adoptive T-cell therapy, and immune checkpoint blockade therapy; radioisotopes such as iodine-131, strontium-89, samarium-153, and radium-223; and radiosensitizing drugs such as Cisplatin, Nimorazole, and Cetuximab, or other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

Nanoclusters and/or ATP can be administered prior to, concurrent with, or subsequent to other agents. If provided at the same time as other agents, the nanoclusters and/or ATP can be provided in the same or in a different composition than the other agents. Thus, nanoclusters and/or ATP and one or more other agents can be presented to the individual by way of concurrent therapy. For example, concurrent therapy may be achieved by administering a dose of a pharmaceutical composition comprising nanoclusters or nanoclusters and ATP and a dose of a pharmaceutical composition comprising at least one other agent, such as another drug for treating an infection or a tumor, which in combination comprise a therapeutically effective dose, according to a particular dosing regimen. Similarly, nanoclusters and/or ATP and one or more other therapeutic agents can be administered in at least one therapeutic dose in separate compositions. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

### Kits

Kits may comprise one or more containers of the compositions described herein comprising a functionalized nanocluster conjugated to a nucleotide (e.g., ATP, dATP, ATPαS, ATPβS, ATPγS, 7-deaza-ATP, or AMP-PCP), or reagents for preparing such compositions, and optionally ATP and/or one or more antibiotics, antifungal agents, or anti-tumor agents. Compositions can be in liquid form or can be lyophilized. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle). The kit can further comprise a container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device. The kit may also provide a delivery device pre-filled with the functionalized nanoclusters.

In addition to the above components, the subject kits may further include (in certain embodiments) instructions for practicing the subject methods (i.e., instructions for treating a subject to eradicate pathogenic cells in a growth arrest phase or tumor cells with nanoclusters, as described herein). These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, Blu-ray, flash drive, and the like, on which the information has been recorded. Yet another form of these instructions that may be present is a website address which may be used via the internet to access the information at a removed site.

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure numbered 1-96 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A method of eradicating a cell in a growth arrest phase, the method comprising contacting the cell in the growth arrest phase with an effective amount of a metallic nanocluster having a size of less than 10 nm, wherein the nanocluster is conjugated to adenosine triphosphate (ATP) or an analogue thereof.
2. The method of aspect 1, wherein the cell is a prokaryotic cell or eukaryotic cell.
3. The method of aspect 2, wherein the cell is a bacterial cell, a fungal cell, or a human cell.
4. The method of aspect 2 or 3, wherein the cell is a benign tumor cell or a malignant tumor cell.
5. The method of aspect any one of aspects 1-4, wherein the nanocluster has a diameter of less than 5 nm.
6. The method of aspect 5, wherein the diameter ranges from about 1 nm to about 5 nm.
7. The method of aspect 6, wherein the diameter is about 2 nm.
8. The method of any one of aspects 1-7, wherein the ATP analogue is selected from the group consisting of ATPαS, ATPβS, ATPγS, deoxyadenosine triphosphate (dATP), 7-deazaadenosine-5′-triphosphate (7-deaza-ATP, and β,γ-methyleneadenosine 5′-triphosphate (AMP-PCP).
9. The method nanocluster of any one of aspects 1-8, wherein the metallic nanocluster comprises a noble metal.
10. The method of aspect 9, wherein the noble metal is gold.
11. The method of any one of aspects 1-10, wherein the nanocluster is conjugated to at least 1000 ATP molecules.
12. A composition for use in a method of treating an infection by bacteria or fungi in a growth arrest phase, the composition comprising a metallic nanocluster having a size of less than 10 nm, wherein the nanocluster is conjugated to adenosine triphosphate (ATP) or an analogue thereof.
13. The composition of aspect 12, further comprising a pharmaceutically acceptable excipient or carrier.
14. The composition of aspect 12 or 13, wherein the nanocluster has a diameter of less than 5 nm.
15. The composition of aspect 14, wherein the diameter ranges from about 1 nm to about 5 nm.
16. The composition of aspect 15, wherein the diameter is about 2 nm.
17. The composition of any one of aspects 12-16, wherein the ATP analogue is selected from the group consisting of ATPαS, ATPβS, ATPγS, deoxyadenosine triphosphate (dATP), 7-deazaadenosine-5′-triphosphate (7-deaza-ATP, and β,γ-methyleneadenosine 5′-triphosphate (AMP-PCP).
18. The composition of any one of aspects 12-17, wherein the metallic nanocluster comprises a noble metal.
19. The composition of aspect 18, wherein the noble metal is gold.
20. The composition of any one of aspects 12-19, wherein the nanocluster is conjugated to at least 1000 ATP molecules.
21. The composition of any one of aspects 12-20, further comprising an antibiotic or an antifungal agent.
22. A method of treating a subject for an infection by bacteria or fungi in a growth arrest phase, the method comprising administering a therapeutically effective amount of the composition any one of aspects 12-21 to the subject.
23. The method of aspects 22, wherein the composition is administered locally at the site of infected tissue.
24. The method of aspect 23, wherein the infection is an ear infection, and the composition is administered locally into the ear canal.
25. The method of aspect 22, wherein the infection is a chronic bacterial or fungal infection.
26. The method of aspect 25, wherein the infection is tuberculosis, cystic fibrosis, a cutaneous wound infection, a urinary tract infection, or a biofilm-associated infection.
27. The method of aspect 26, wherein the biofilm-associated infection is a catheter associated infection, a central line-associated infection, an endotracheal tube associated infection, an implantable device-associated infection, or a prosthetic joint-associated infection.
28. The method of any one of aspects 22-27, further comprising administering a therapeutically effective amount of at least one antibiotic or antifungal agent to the subject.
29. The method of any one of aspects 22-28, wherein multiple cycles of treatment are administered to the subject.
30. The method of any one of aspects 22-29, wherein the bacteria are Gram-negative bacteria.
31. A composition for use in a method of treating cancer, the composition comprising a metallic nanocluster having a size of less than 10 nm, wherein the nanocluster is conjugated to adenosine triphosphate (ATP) or an analogue thereof.
32. The composition of aspect 31, further comprising a pharmaceutically acceptable excipient or carrier.
33. The composition of aspect 31 or 32, further comprising an anti-cancer agent.
34. The composition of any one of aspects 31-33, wherein the metallic nanocluster comprises a noble metal.
35. The composition of aspect 34, wherein the noble metal is gold.
36. The composition of any one of aspects 31-35, wherein the nanocluster is conjugated to at least 1000 ATP molecules.
37. The composition of any one of aspects 37-39, wherein the cancer is melanoma or schwannoma.
38. A method of treating cancer in a subject, the method comprising administering a therapeutically effective amount of the composition of any one of aspects 31-37 to the subject.
39. The method of aspect 38, wherein the composition is administered locally, intratumorally, intravenously, subcutaneously, by inhalation, or topically.
40. The method of aspect 38, wherein the composition is administered locally to a tumor.
41. The method of any one of aspects 38-40, wherein multiple cycles of treatment are administered to the subject.
42. The method of any one of aspects 38-41, further comprising administering a therapeutically effective amount of ATP to the subject.
43. The method of any one of aspects 38-42, wherein the cancer is melanoma or schwannoma.
44. The method of any one of aspects 38-43, wherein the nanocluster has a diameter of less than 5 nm.
45. The method of aspect 44, wherein the diameter ranges from about 1 nm to about 5 nm.
46. The method of aspect 45, wherein the diameter is about 2 nm.
47. The method of any one of aspects 38-46, wherein the ATP analogue is selected from the group consisting of ATPαS, ATPβS, ATPγS, deoxyadenosine triphosphate (dATP), 7-deazaadenosine-5′-triphosphate (7-deaza-ATP, and β,γ-methyleneadenosine 5′-triphosphate (AMP-PCP).
48. The method nanocluster of any one of aspects 38-47, wherein the metallic nanocluster comprises a noble metal.
49. The method of aspect 48, wherein the noble metal is gold.
50. The method of any one of aspects 38-49, wherein the nanocluster is conjugated to at least 1000 ATP molecules.
51. A method of treating melanoma in a subject, the method comprising administering to the subject a therapeutically effective amount of ATP in combination with a therapeutically effective amount of a metallic nanocluster having a size of less than 10 nm, wherein the nanocluster is conjugated to adenosine triphosphate (ATP) or an analogue thereof.
52. The method of aspect 51, wherein the ATP and the metallic nanocluster are administered intratumorally or topically.
53. The method of aspect 51 or 52, wherein the nanocluster has a diameter of less than 5 nm.
54. The method of anyone of aspects 51-53, wherein the diameter ranges from about 1 nm to about 5 nm.
55. The method of aspect 54, wherein the diameter is about 2 nm.
56. The method of any one of aspects 51-55, wherein the ATP analogue is selected from the group consisting of ATPαS, ATPβS, ATPγS, deoxyadenosine triphosphate (dATP), 7-deazaadenosine-5′-triphosphate (7-deaza-ATP, and β,γ-methyleneadenosine 5′-triphosphate (AMP-PCP).
57. The method nanocluster of any one of aspects 51-56, wherein the metallic nanocluster comprises a noble metal.
58. The method of aspect 57, wherein the noble metal is gold.
59. The method of any one of aspects 51-58, wherein the nanocluster is conjugated to at least 1000 ATP molecules.
60. A method of inhibiting a FtsH protease, the method comprising contacting the FtsH protease with a metallic nanocluster having a size of less than 10 nm, wherein the nanocluster is conjugated to adenosine triphosphate (ATP) or an analogue thereof, wherein the protease activity of the FtsH protease is inhibited.
61. The method of aspect 60, wherein the nanocluster has a diameter of less than 5 nm.
62. The method of aspect 61, wherein the diameter ranges from about 1 nm to about 5 nm.
63. The method of aspect 62, wherein the diameter is about 2 nm.
64. The method of any one of aspects 60-63, wherein the ATP analogue is selected from the group consisting of ATPαS, ATPβS, ATPγS, deoxyadenosine triphosphate (dATP), 7-deazaadenosine-5′-triphosphate (7-deaza-ATP, and β,γ-methyleneadenosine 5′-triphosphate (AMP-PCP).
65. The method nanocluster of any one of aspects 60-64, wherein the metallic nanocluster comprises a noble metal.
67. The method of aspect 65, wherein the noble metal is gold.
66. The method of any one of aspects 60-67, wherein the nanocluster is conjugated to at least 1000 ATP molecules.
68. A method of inhibiting a purinergic P2X7 receptor (P2X7R), the method comprising contacting the P2X7R with a metallic nanocluster having a size of less than 10 nm, wherein the nanocluster is conjugated to adenosine triphosphate (ATP) or an analogue thereof, wherein the activity of the P2X7R is inhibited.
69. A method of increasing phagocytic clearance in a tissue, the method comprising contacting the tissue with a metallic nanocluster having a size of less than 10 nm, wherein the nanocluster is conjugated to adenosine triphosphate (ATP) or an analogue thereof, wherein the phagocytic clearance is increased in the tissue.
70. A method of reducing NLRP3 activation and IL-1beta-mediated inflammation in a subject, the method comprising administering a therapeutically effective amount of a metallic nanocluster having a size of less than 10 nm to the subject, wherein the nanocluster is conjugated to adenosine triphosphate (ATP) or an analogue thereof.
71. The method of aspect 70, wherein said administering the therapeutically effective amount of the metallic nanocluster reduces microglial inflammation, reduces oxidative stress, and increases phagocytic clearance in the subject.
72. The method of aspect 70 or 71, wherein the nanocluster has a diameter of less than 5 nm.
73. The method of aspect 72, wherein the diameter ranges from about 1 nm to about 5 nm.
74. The method of aspect 73, wherein the diameter is about 2 nm.
75. The method of any one of aspects 70-74, wherein the ATP analogue is selected from the group consisting of ATPαS, ATPβS, ATPγS, deoxyadenosine triphosphate (dATP), 7-deazaadenosine-5′-triphosphate (7-deaza-ATP, and β,γ-methyleneadenosine 5′-triphosphate (AMP-PCP).
76. The method nanocluster of any one of aspects 70-75, wherein the metallic nanocluster comprises a noble metal.
77. The method of aspect 76, wherein the noble metal is gold.
78. The method of any one of aspects 70-77, wherein the nanocluster is conjugated to at least 1000 ATP molecules.
79. A method of inducing endoplasmic reticulum (ER) stress in a cell, the method comprising contacting the cell with an effective amount of a metallic nanocluster having a size of less than 10 nm, wherein the nanocluster is conjugated to adenosine triphosphate (ATP) or an analogue thereof.
80. The method of aspect 79, wherein the cell is a cancerous cell.
81. The method of aspect 79 or 80, wherein the nanocluster has a diameter of less than 5 nm.
82. The method of aspect 81, wherein the diameter ranges from about 1 nm to about 5 nm.
83. The method of aspect 82, wherein the diameter is about 2 nm.
84. The method of any one of aspects 79-83, wherein the ATP analogue is selected from the group consisting of ATPαS, ATPβS, ATPγS, deoxyadenosine triphosphate (dATP), 7-deazaadenosine-5′-triphosphate (7-deaza-ATP, and β,γ-methyleneadenosine 5′-triphosphate (AMP-PCP).
85. The method nanocluster of any one of aspects 79-84, wherein the metallic nanocluster comprises a noble metal.
86. The method of aspect 85, wherein the noble metal is gold.
87. The method of any one of aspects 79-86, wherein the nanocluster is conjugated to at least 1000 ATP molecules.
88. A method of inhibiting proliferation of a cancerous cell, the method comprising contacting the cell with an effective amount of a metallic nanocluster having a size of less than 10 nm, wherein the nanocluster is conjugated to adenosine triphosphate (ATP) or an analogue thereof.
89. The method of aspect 88, wherein the cancerous cell is a melanoma or schwannoma cell.
90. The method of aspect 88 or 89, wherein the nanocluster has a diameter of less than 5 nm.
91. The method of aspect 90, wherein the diameter ranges from about 1 nm to about 5 nm.
92. The method of aspect 91, wherein the diameter is about 2 nm.
93. The method of any one of aspects 88-92, wherein the ATP analogue is selected from the group consisting of ATPαS, ATPβS, ATPγS, deoxyadenosine triphosphate (dATP), 7-deazaadenosine-5′-triphosphate (7-deaza-ATP, and β,γ-methyleneadenosine 5′-triphosphate (AMP-PCP).
94. The method nanocluster of any one of aspects 88-93, wherein the metallic nanocluster comprises a noble metal.
95. The method of aspect 94, wherein the noble metal is gold.
96. The method of any one of aspects 88-95, wherein the nanocluster is conjugated to at least 1000 ATP molecules.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Targeting ATP-Dependent Proteases and Magnesium Homeostasis in Bacteria

We propose a novel strategy for targeting bacterial cells during their growth arrest phase. In chronic disease, bacteria are initially refractory to complete killing by antimicrobials, not because of resistance but due to their dormancy during growth arrest. Failure to eliminate these cells contributes to the relapse observed in many chronic bacterial infections currently managed with cycles of antibiotics. There is an unmet medical to develop treatments that can kill bacteria cells during their growth arrest phase.

Here, we describe new agents to eradicate bacteria in the growth arrest phase. The lack of a suitable animal model that replicates human relapsing chronic infection has hindered this goal. The disease chronic suppurative otitis media (CSOM) exemplifies this problem, afflicting 330 million worldwide. CSOM is a chronically discharging infected middle ear most frequently caused by the Gram-negative Pseudomonas aeruginosa (P. aeruginosa), resulting in permanent hearing loss and surgery. Our lab recently created and validated a novel relapsing P. aeruginosa CSOM animal model with bioluminescent real-time tracking. We have shown that it mimics the human condition, relapses following topical fluoroquinolone therapy and is stable for over 6 months. Our model now allows the testing of novel therapeutic strategies to prevent relapse in chronic bacterial infections and evaluate the potential for antibiotic resistance development.

Motivated by observations on nanoparticle-bacteria interactions, we began exploring interactions with gold nanoclusters, observing performance in various growth phases and in biofilms. One candidate, a gold nanocluster functionalized with adenosine triphosphate (AuNC@ATP) displayed excellent activity with sterilization of planktonic cultures (10⁸ CFU/mL) and biofilms of extensively drug-resistant (XDR) E. coli, K. pneumonia and P. aeruginosa. AuNC@ATP provides a novel antimicrobial strategy at its uniquely small size (2.42±0.43 nm).

Recent work shows magnesium (Mg²⁺), instead of other ions or nutrient components, is a critical element for bacteria to become refractory to antibiotic killing. Bacteria cope with low cytosolic Mg²⁺ during the growth arrest phase by reducing the Mg²⁺ chelating ATP level and ribosome production. This protects cytoplasmic Mg²⁺ for essential cellular processes. Having Mg²⁺ and ATP balance at much lower than normal levels, provides the opportunity to “tip the scale” to cause death. Driving ATP levels up would send intracellular Mg²⁺ levels to below critical. Although not wishing to be bound by theory, we hypothesize that the presence of AuNC@ATP in the cytoplasm increases the Mg²⁺ chelating ATP amount to a non-physiological level and collapses the cytoplasmic pool of free Mg²⁺, causing bacterial cell death during the growth arrest phase. AuNC@ATP may also work through a second mechanism involving inhibition of proteolytic activity. Degradation of misfolded and nonfunctional proteins by ATP-dependent proteases continues unabated during cytoplasmic Mg²⁺ limitation. A reduction in the amount of ATP is necessary to stabilize substrates of ATP-dependent proteases. The FtsH proteases are inner membrane ATP-dependent proteases that serve as crucial protein quality inspectors by digesting misassembled or damaged membrane proteins and short-lived water-soluble enzymes and transcription factors. ATP hydrolysis and exchange within the nucleotide pockets of FtsH are primarily responsible for inducing the conformational rearrangements required for substrate processing. The FtsH is closed during the ATP hydrolysis but operates in the open form. Therefore, high ATP conditions (by AuNC@ATP) may obstruct FtsH binding pocket access, enforcing a closed state, inhibiting proteolytic activity.

### AuNC@ATP Mediated Cell Death Through the Disruption of Mg²⁺ Homeostasis

AuNC@ATP can be used to deplete cytoplasmic free [Mg²⁺]. We correlate the time of bacterial death and the cytoplasmic free [Mg²⁺] change. Manipulation of cytoplasmic Mg²⁺ can be used as a strategy to kill dormant bacteria.

### Inhibition of the FtsH Proteases During AuNC@ATP-Mediated Cell Death

We confirm the requirement of functional FtsH for AuNC@ATP activity in Gram-negative bacteria. Other ATP-dependent proteases, including ClpP and the Lon proteases, present in many Gram-negative bacteria, are also involved in the proteolysis of defective and misfolded proteins. Manipulation of cytoplasmic ATP levels using AuNC@ATP can be used as a strategy to kill dormant bacteria.

### Example 2

### Antitumoral Activity of AuNC@ATP

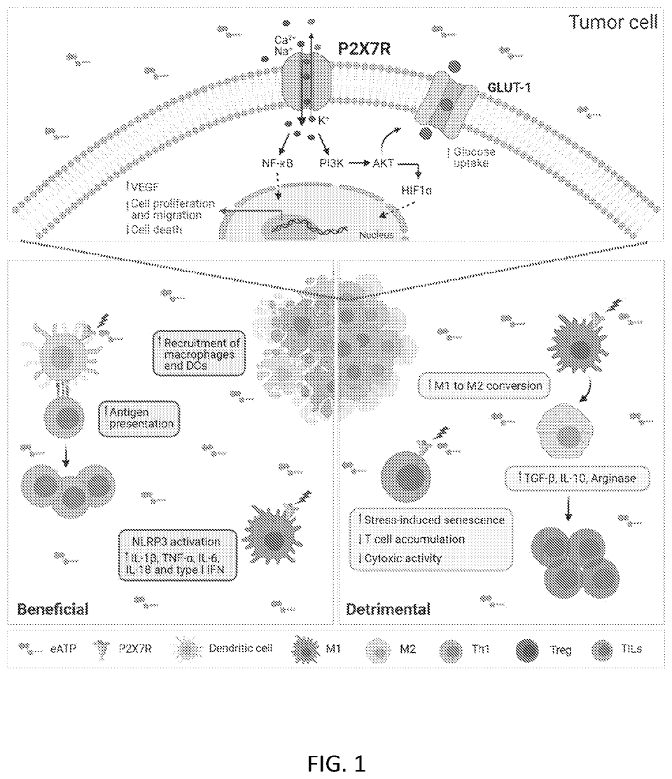

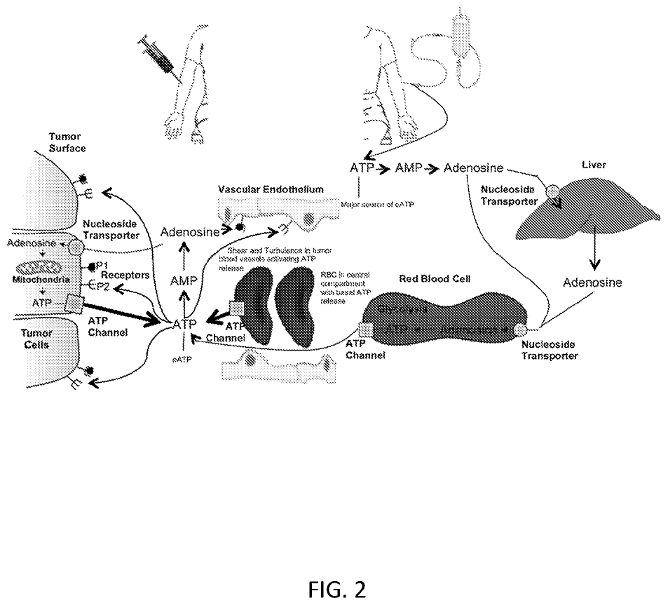

Adenosine triphosphate (ATP) is one of the main biochemical components released into the tumor microenvironment (TME) in a non-regulated or regulated fashion. P2X7R is widely expressed in the different cell types constituting the TME (FIG. 1) (Grassi et al. (2021) Front. Cell Dev. Biol. 9:694831). Extracellular ATP (eATP) can promote immunosuppression or support antitumor immunity depending on its concentration and specific receptors expressed by immune and cancer cells (Vultaggio-Poma et al. (2020) Cells 9(11):2496).

Due to its crucial role in the tumor-host interaction, the most obvious target for cancer therapy would be eATP itself. Therefore, further increasing the eATP concentration can be used to exploit ATP-dependent cytotoxicity. Without wishing to be bound by theory, the ATP-binding receptor, P2X7, responds to high levels of ATP by triggering a cascade of reactions that results in ATP-dependent cytotoxicity. Reports on the anticancer activity of ATP have accumulated over the years. For example, it has been shown that treatment of human tumor cells with ADP or ATP yields arrest of growth in the S phase of the cell cycle (Rapaport (1983) J. Cell. Physiol. 114(3):279-83). Furthermore, experimental cancer therapy in mice by ATP revealed that intraperitoneal injection of ATP (50 mM) into tumor-bearing mice effectively reduced tumor size (Rapaport (1988) Eur. J. Cancer Clin. Oncol. 24(9):1491-7). Other in vivo studies performed in human prostate cancer xenografts have confirmed this observation by showing that daily intraperitoneal administration of ATP (25 mM) causes a significant tumor regression (Shabbir et al. (2008) BJU Int. 102(1):108-12). Finally, tumor engraftment in P2X7R null mice showed a lack of inflammatory infiltration and accelerated tumor progression, suggesting the host immune system benefits from ATP-mediated activation of P2X7R for controlling tumor growth (Adinolfi et al. (2015) Cancer Res. 75(4):635-44). Conversely, pharmacological blockade of P2X7R with A740003 showed an opposite effect on tumor outcome (Adinolfi et al., supra). However, despite the beneficial effect of exogenously administered ATP for cancer treatment, its major disadvantage is the rapid degradation by ectonucleotidases (i.e., ectoenzyme). Therefore, high doses of ATP are required to reach a therapeutic effect. The pharmacokinetics of ATP was investigated in a clinical trial that included 15 patients with advanced malignancies (solid tumors) (Rapaport et al. (2015) Purinergic Signal 11(2):251-62). The ATP (50-100 μg/kg min of ATP) was administered to the patients by continuous intravenous infusion for 8 h once weekly for 8 weeks. The data suggest that a significant fraction of the total exogenously administered ATP is sequestered into the intracellular compartments of the erythrocytes after an 8 h intravenous infusion. Rapid degradation of intravenously administered ATP to adenosine and subsequent accumulation of ATP inside erythrocytes indicate the existence of very effective mechanisms for uptake of adenosine from blood plasma.

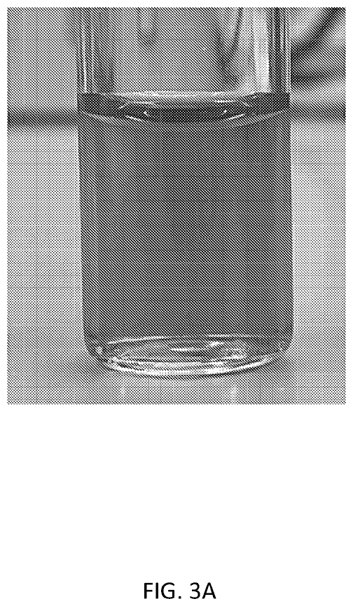

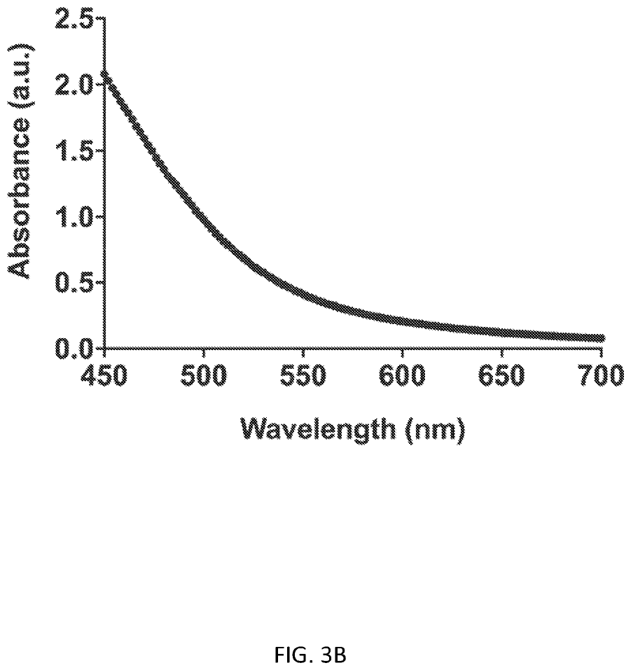

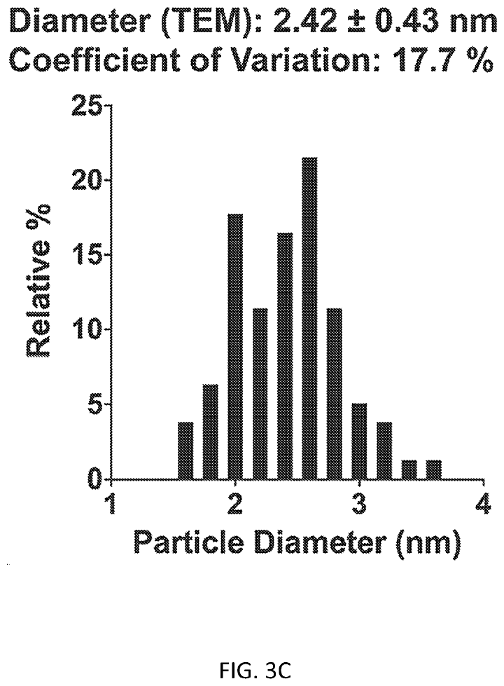

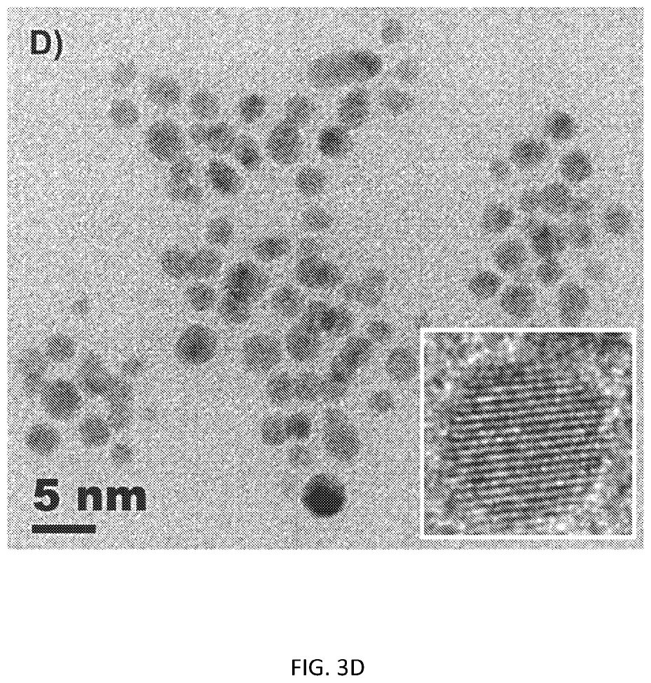

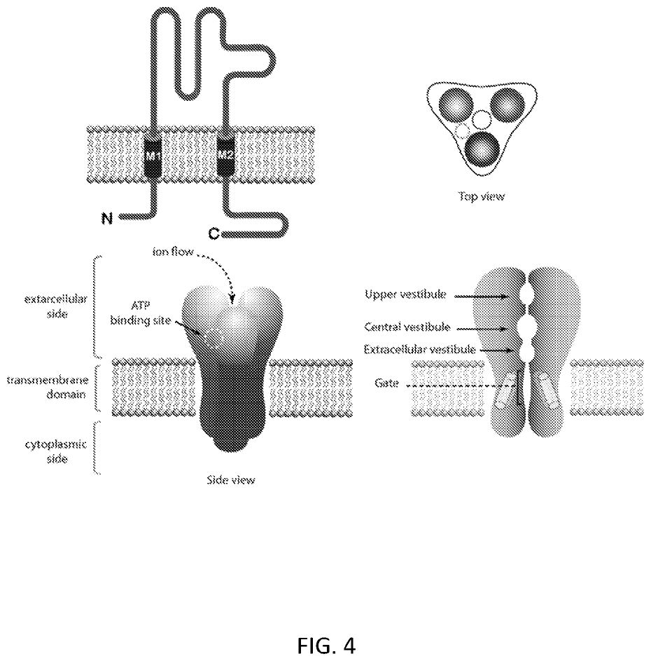

Gold nanoclusters (AuNCs) provide attractive candidates for ATP delivery. AuNCs could protect ATP from being degraded by ectoenzyme and prevent excess adenosine generation. We recently engineered an ultrasmall gold nanocluster functionalized with adenosine triphosphate (AuNC@ATP) (FIG. 3). Each AuNC@ATP contains 1000 molecules of ATP. Therefore, an alternative strategy to increase eATP levels in the TME is a nanoformulation that prevents premature degradation of ATP (AuNC@ATP or preparation of liposomes containing AuNC@ATP).

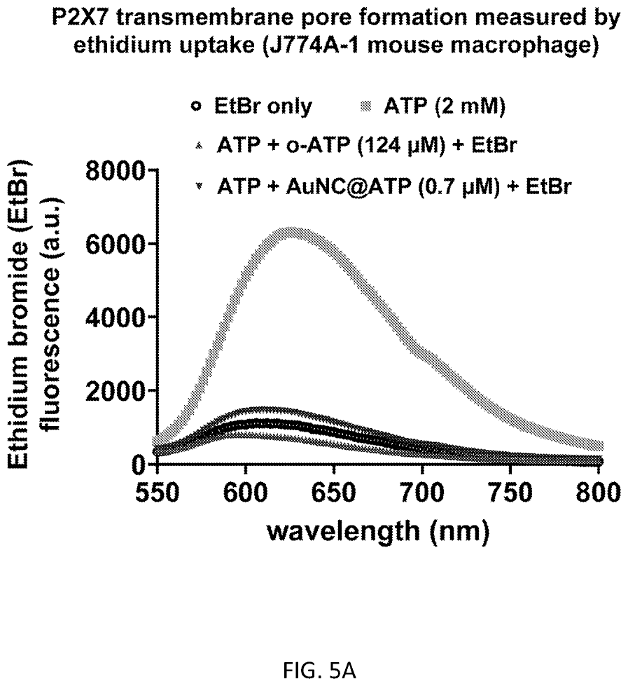

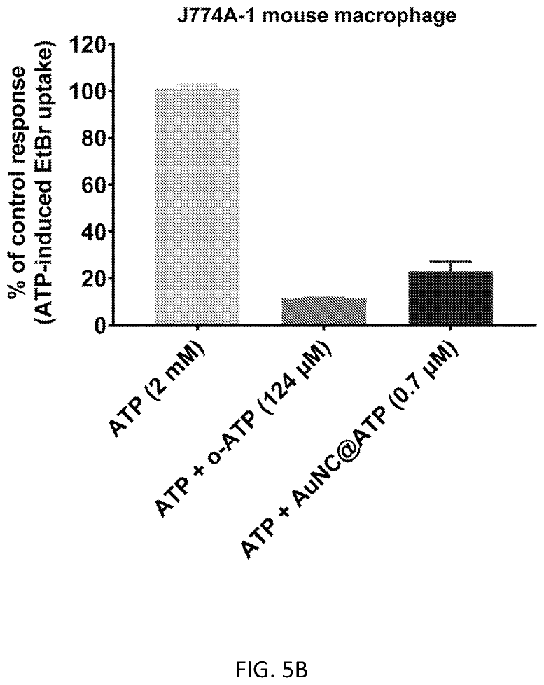

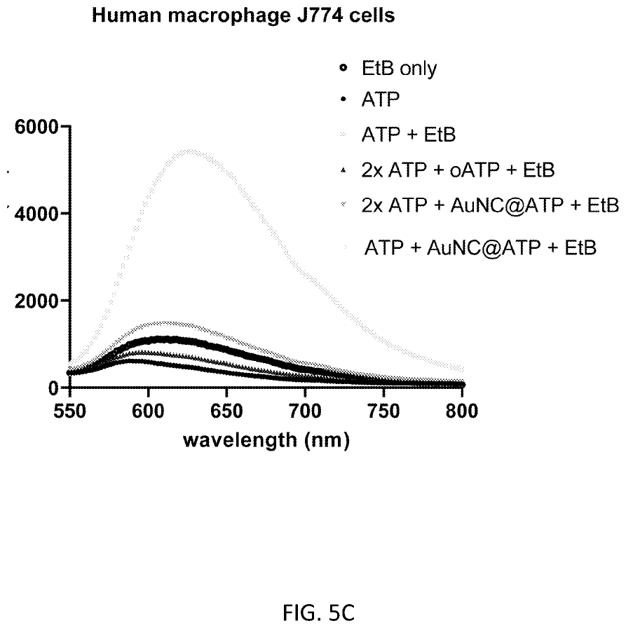

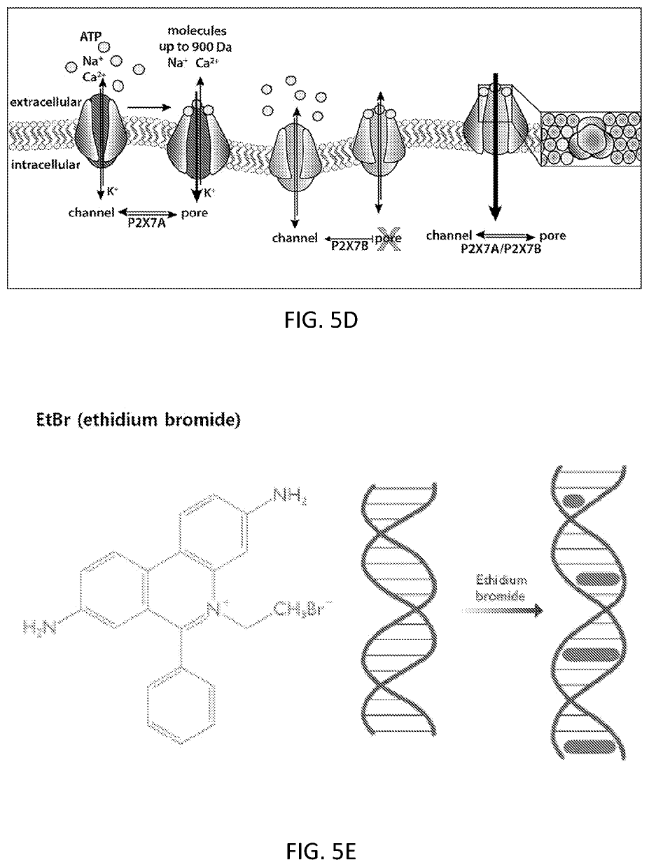

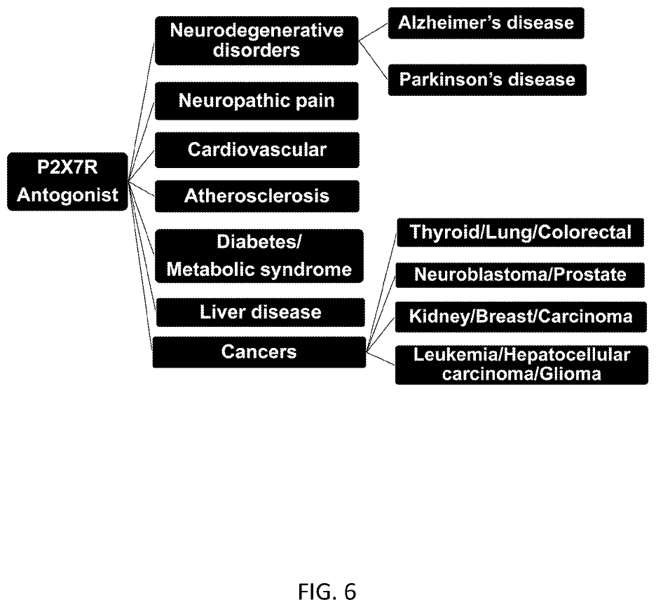

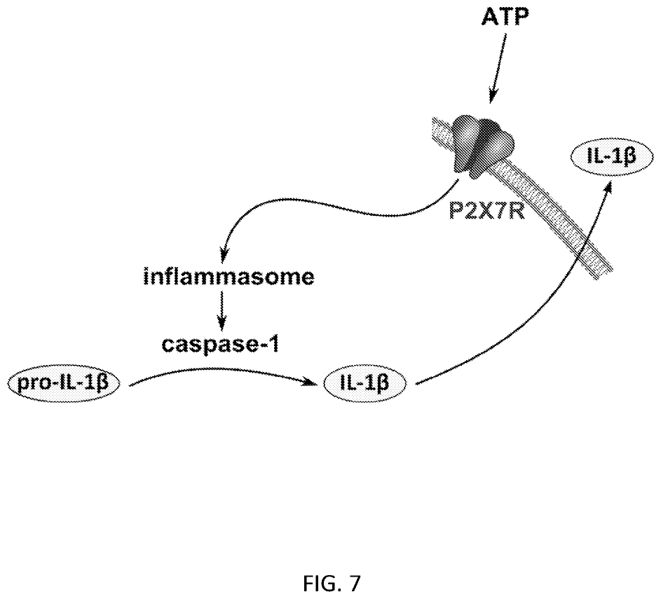

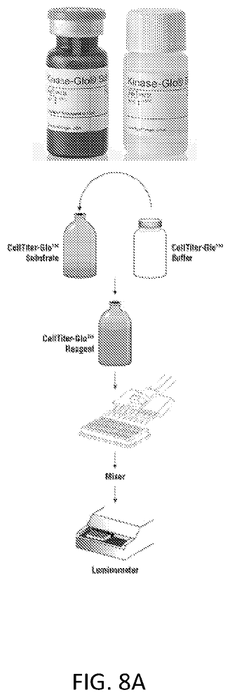

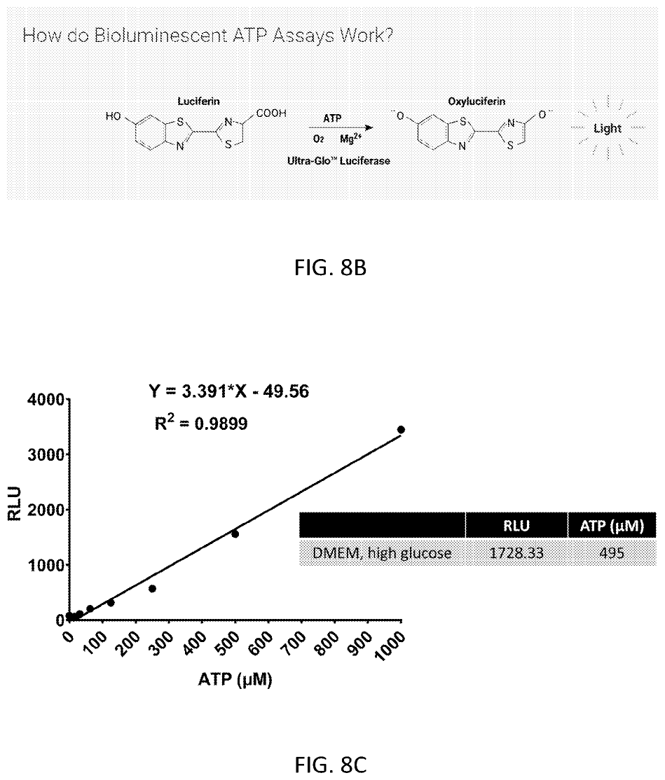

As shown in FIGS. 5A-5E, AuNC@ATP acts as an antagonist of P2X7 as determined by measuring ATP-induced ethidium uptake in J774A-1 macrophages. The activation of the P2X7 receptor with high concentrations of adenosine triphosphate (ATP>0.05 mM) results in the formation of a large transmembrane pore permeable to molecules up to 900 Da such as Ethidium bromide (EtBr). Therefore, the inhibition of the P2X7 activation (i.e., pore formation) was quantified as the reduction of ATP-induced ethidium uptake in J774A-1 macrophages expressing. The EtBr uptake was induced in J774A-1 by adding ATP (2 mM). The antagonists of the P2X7 receptor (i.e., O-ATP and AuNC@ATP) and ATP were added at the same time. Then cells were incubated for 1 h at 37° C. before measuring the fluorescence of ethidium bromide.

### Expression of P2X7 Across Cancer Types

### Prostate Cancer:

An immunohistochemistry (IHC) study looking at 116 prostate cancer biopsies using an affinity-purified polyclonal antibody to the E200 epitope of P2X7 (supplied by Biosceptre) has shown that P2X7 was present in all malignant samples regardless of their stage or the age of the patient (Slater et al. (2004) Histopathology 44(3):206-15). Furthermore, functional P2X7 was shown to drive invasion and metastasis of prostate cancer cell lines stimulated by extracellular ATP (Ghalali et al. (2014) Carcinogenesis 35(7):1547-55).

### Lung Cancer:

P2X7 is expressed in human NSCLC cell lines, including A549, PC9 and H292 cells but not in the nonmalignant bronchial epithelial cells BEAS-2B.

### Kidney Cancer:

Clear-cell renal cell carcinoma (ccRCC) is the most common form of renal cell carcinoma. In a study analyzing 273 ccRCC patients by IHC, P2X7 expression was correlated with the clinicopathologic features and cancer-specific survival (CSS) (Liu et al. (2015) Cancer Sci. 106(9):1224-31)

### Cutaneous Squamous-Cell and Basal-Cell Carcinomas:

P2X7 is highly expressed both in nodular basal cell carcinomas (BCC) and in infiltrative BCC cells, which were shown to be present in some tumor cell nuclei (PMID: 12880424). In addition, P2X7 expression was also found in the human squamous cell carcinoma (SCC) cell line A431 (Greig et al. (2003) J. Invest. Dermatol. 121(2):315-27).

### Mitochondrial ATP-Dependent Proteases as Potential Anticancer Targets

Emerging studies suggest the therapeutic potential of targeting the matrix ATP-dependent protease ClpXP (Feng et al. (2020) Cancers (Basel) 13(9):2020) and Lon (Bernstein et al. (2021) Blood 119(14):3321-9). Therefore, we hypothesize that AuNC@ATP could act as an inhibitor of ATP-dependent proteases (ClpXP and Lon). Therefore, the potential antitumoral activity of AuNC@ATP could be mediated by the activation of P2X7 and the inhibition of mitochondrial ATP-dependent proteases.

### Example 3

### Exploiting Adenosine Triphosphate Coated-Coated Gold Nanoclusters to Induce Endoplasmic Reticulum Stress to Block Cancer Growth

### Introduction

The endoplasmic reticulum (ER) is an essential organelle where secreted and transmembrane proteins are synthesized, folded and modified (PMID: 26433683). Disrupting the protein-folding capacity of this organelle provokes ER stress characterized by the buildup of misfolded or unfolded proteins. This accumulation of unfolded and misfolded proteins activates the unfolded protein response (UPR), which works in various ways to restore the balance between the ER's folding ability and the amount of unfolded proteins (PMID: 19609758; PMID: 33214692). On the other hand, extreme ER stress caused by the uncontrolled accumulation of unfolded and misfolded proteins in this organelle can lead to a terminal UPR that induces cell death (PMID: 33214692).

Cancer cells grow while facing different stresses like lack of oxygen, low nutrients, and DNA damage. They adapt to these challenges by using the UPR. However, in most normal cells, which usually do not experience such stress, the UPR pathways are not active (PMID: 19861963). Because the UPR is crucial for diseases like cancer to persist, it is worth investigating if it can be employed in treatment. One strategy involves using ER stress-inducing agents as anti-cancer therapies to create severe ER stress in tumour tissues (PMID: 36345017). For instance, bortezomib is the first approved proteasome inhibitor drug for the clinical treatment of cancer and acts by causing excessive accumulation of aberrant proteins, which augments ER stress, leading to the death of malignant cells (PMID: 26026090; PMID: 18818117; PMID: 20133382). Furthermore, bortezomib and dipyridamole worked with effective synergy to enhance ER stress in treated cancer cells (PMID: 25245324).

In our earlier work on developing a new antimicrobial agent to eliminate bacteria resistant to multiple drugs, we engineered adenosine triphosphate coated-coated gold nanoclusters (AuNC@ATP). Unlike typical antibiotics focusing on particular cell processes, AuNC@ATP fights bacteria by causing an accumulation of aberrant proteins in the periplasm. Considering how AuNC@ATP combats microbes, we hypothesized that it could be beneficial as an agent to induce ER stress for inhibiting cancer growth.

### Results and Discussion

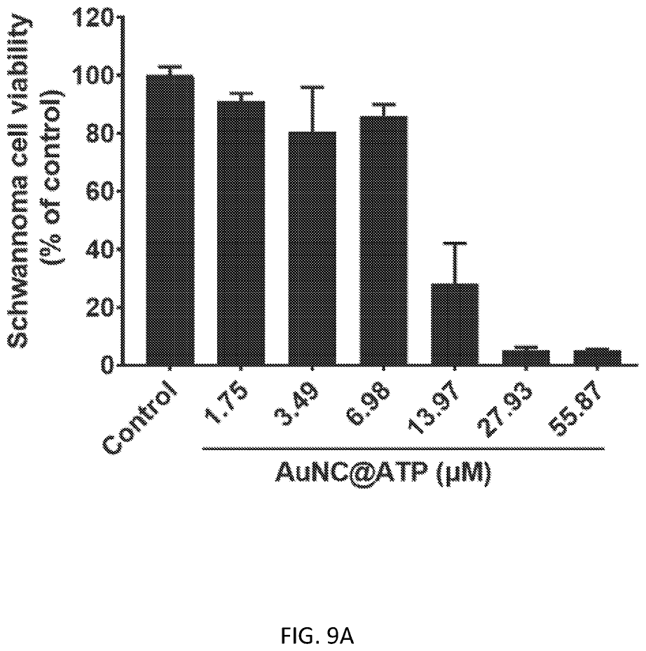

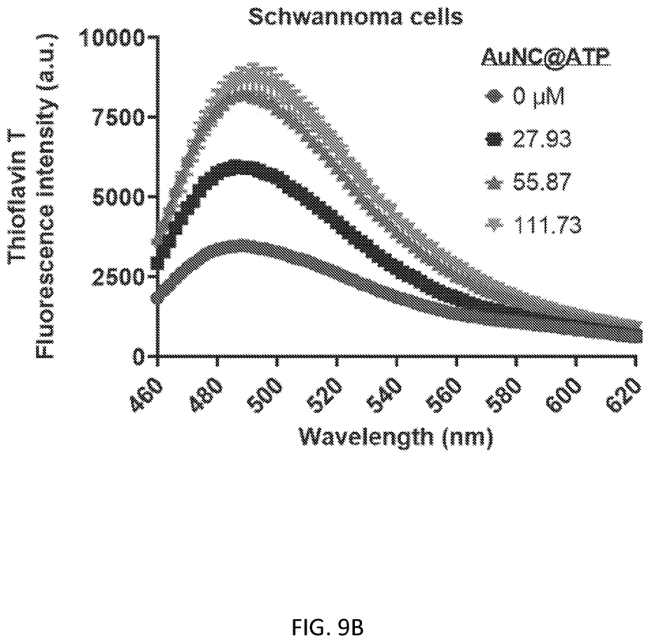

AuNC@ATP induces cell death and ER stress in schwannoma cells. We first elucidate the effects of AuNC@ATP on the schwannoma cell line. We exposed schwannoma cells to various AuNC@ATP concentrations for 24 hours and assessed cell viability using an MTT assay. The results showed that AuNC@ATP caused a decrease in cell viability, which was directly related to its concentration. A concentration of 27.93 μM led to a substantial loss of cell viability, reaching 95% (FIG. 9A). We then measured the ER stress caused by AuNC@ATP in schwannoma cells. Thioflavin T (Th-T) is a small molecule that becomes more fluorescent when it binds to protein aggregates (PMID: 23747341; PMID: 31617349). The enhanced Th-T fluorescence is directly connected to established signs of UPR activation. Therefore, we employed Th-T to detect ER stress induced by AuNC@ATP. AuNC@ATP caused a Th-T fluorescence increase in schwannoma cells, which was linked to the concentration of AuNC@ATP (FIG. 9B). These data suggest that AuNC@ATP could be beneficial as an agent to induce ER stress for inhibiting cancer growth.

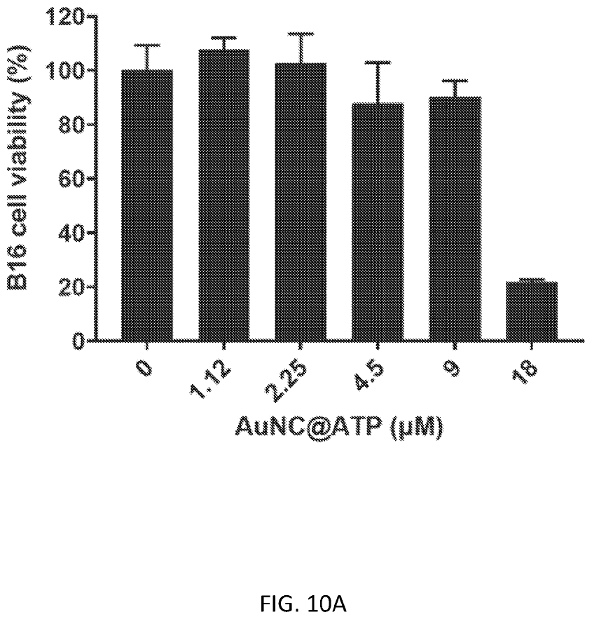

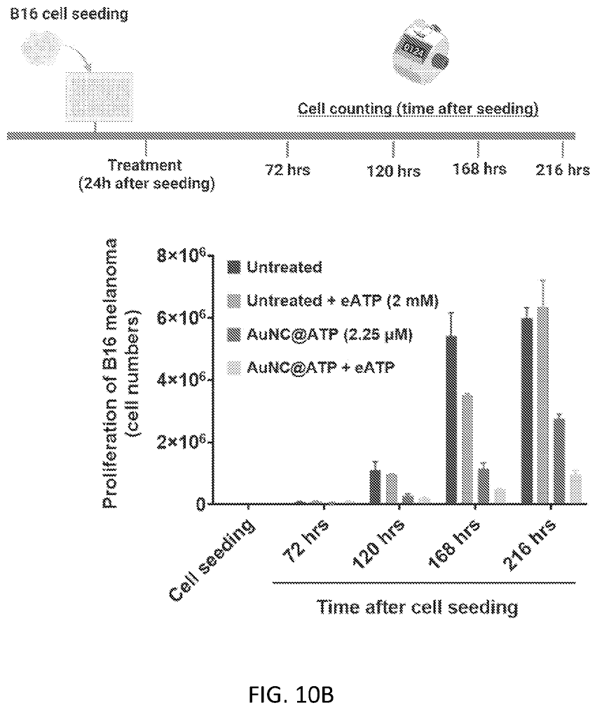

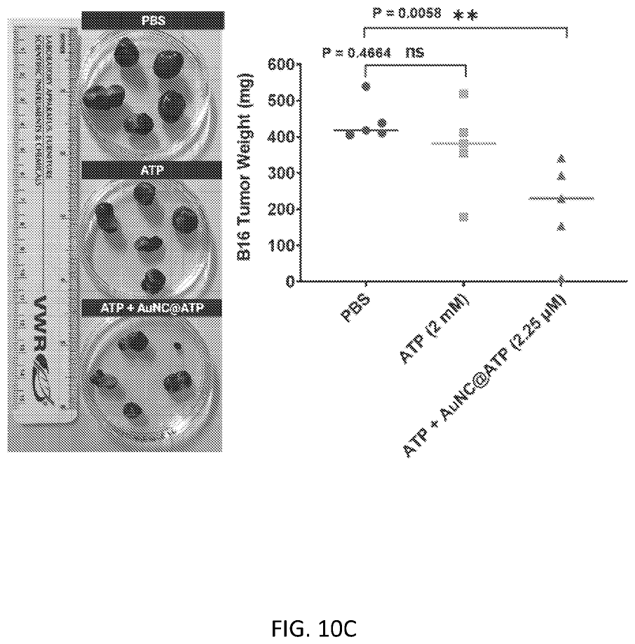

AuNC@ATP slows the growth of melanoma cells and hinders tumor development in a xenograft model. We first elucidate the effects of AuNC@ATP on the B16-F10 melanoma cell line. The findings indicated that as the concentration of AuNC@ATP increased, the viability of B16-F10 cells decreased. Cell viability was significantly reduced at a concentration of 18 μM, reaching 80% (FIG. 10A). Next, we wanted to see if AuNC@ATP would impact the growth of B16-F10 melanoma cells at concentrations below those that cause cell death. We cultured B16-F10 cells in a medium containing a sub-lethal concentration of AuNC@ATP (2.25 μM) and monitored cell growth at various intervals. We compared the cell count at each time point with B16-F10 cells growing in a medium without AuNC@ATP (Control culture). We also included free ATP as a reference for comparison. Our data demonstrates that a sub-lethal concentration of AuNC@ATP noticeably retards the growth of B16-F10 cells. After 168 hrs, these cells reached 1×10⁶ when exposed to AuNC@ATP, compared to 6×10⁶ when growing without it (FIG. 10B). It is important to note that even at a concentration approximately 888 times greater than AuNC@ATP, free ATP (eATP, 2 mM) does not achieve the same level of effectiveness in inhibiting B16-F10 cell proliferation (FIG. 10B). This data indicates that the complete AuNC@ATP entity was necessary to inhibit B16-F10 cell proliferation.

Interestingly, when AuNC@ATP and free ATP were combined, they demonstrated a synergistic effect in inhibiting B16-F10 cell proliferation (FIG. 10B). The ATP levels outside of cells (extracellular ATP or eATP) are significantly higher in the tumor microenvironment (TME) compared to normal tissues, and it is carefully controlled in healthy tissues (PMID: 35738682). Indeed, previous research measured the eATP concentration in the tumor microenvironment of mice with B16F10 tumors using the pmeLuc probe. They found that when there was a lack of nutrients, there was a significant increase in eATP, along with a substantial release of microparticles containing ATP and naked mitochondria. This is likely a primary mechanism responsible for the buildup of eATP in the TME (PMID: 34976217). Considering these discoveries and the observed in vitro synergistic effect between AuNC@ATP and eATP in inhibiting B16-F10 cell proliferation, it is reasonable to assume that AuNC@ATP would perform effectively in a tumor TME with increased levels of eATP.

Surgical excision is the preferred treatment for most patients with localized cutaneous melanoma, and it is often curative. Nonetheless, in some cases, patients may later experience a recurrence of the disease that has spread. Adjuvant therapy is crucial in treating stage III and IV melanoma following surgical removal (PMID: 34503725). Clinical trials for neoadjuvant therapies are currently underway. These trials are particularly advantageous for treating diseases that can be surgically removed and have a high risk of recurrence. Therefore, AuNC@ATP is envisioned to be used as both adjuvant and neoadjuvant therapies for melanoma. These treatments have the potential to offer substantial advantages to patients who face the highest risk of disease recurrence after surgical removal. Given the growing interest in intratumoral delivery of neoadjuvant agents for treating solid tumors in their earlier stages (PMID: 32071116), we evaluated whether the combination of AuNC@ATP and eATP could hinder the B16-F10 melanoma tumor development in a xenograft model. Our findings indicate that the intratumoral injection of a combination of AuNC@ATP and eATP once daily for 14 days substantially reduces tumor growth in vivo (FIG. 10B). This supports the potential use of this nanocluster as a neoadjuvant therapy for melanoma.

What is claimed is:

1. A method of eradicating a cell in a growth arrest phase, the method comprising contacting the cell in the growth arrest phase with an effective amount of a metallic nanocluster having a size of less than 10 nm, wherein the nanocluster is conjugated to adenosine triphosphate (ATP) or an analogue thereof.

2. The method of claim 1, wherein the cell is a prokaryotic cell or eukaryotic cell.

3. The method of claim 2, wherein the cell is a bacterial cell, a fungal cell, or a human cell.

4. The method of claim 2 or 3, wherein the cell is a benign tumor cell or a malignant tumor cell.

5. The method of claim any one of claims 1-4, wherein the nanocluster has a diameter of less than 5 nm.

6. The method of claim 5, wherein the diameter ranges from about 1 nm to about 5 nm.

7. The method of claim 6, wherein the diameter is about 2 nm.

8. The method of any one of claims 1-7, wherein the ATP analogue is selected from the group consisting of ATPαS, ATPβS, ATPγS, deoxyadenosine triphosphate (dATP), 7-deazaadenosine-5′-triphosphate (7-deaza-ATP, 5′- and β,γ-methyleneadenosine triphosphate (AMP-PCP).

9. The method nanocluster of any one of claims 1-8, wherein the metallic nanocluster comprises a noble metal.

10. The method of claim 9, wherein the noble metal is gold.

11. The method of any one of claims 1-10, wherein the nanocluster is conjugated to at least 1000 ATP molecules.

12. A composition for use in a method of treating an infection by bacteria or fungi in a growth arrest phase, the composition comprising a metallic nanocluster having a size of less than 10 nm, wherein the nanocluster is conjugated to adenosine triphosphate (ATP) or an analogue thereof.

13. The composition of claim 12, further comprising a pharmaceutically acceptable excipient or carrier.

14. The composition of claim 12 or 13, wherein the nanocluster has a diameter of less than 5 nm.

15. The composition of claim 14, wherein the diameter ranges from about 1 nm to about 5 nm.

16. The composition of claim 15, wherein the diameter is about 2 nm.

17. The composition of any one of claims 12-16, wherein the ATP analogue is selected from the group consisting of ATPαS, ATPβS, ATPγS, deoxyadenosine triphosphate (dATP), 7-deazaadenosine-5′-triphosphate (7-deaza-ATP, and β,γ-methyleneadenosine 5′-triphosphate (AMP-PCP).

18. The composition of any one of claims 12-17, wherein the metallic nanocluster comprises a noble metal.

19. The composition of claim 18, wherein the noble metal is gold.

20. The composition of any one of claims 12-19, wherein the nanocluster is conjugated to at least 1000 ATP molecules.

21. The composition of any one of claims 12-20, further comprising an antibiotic or an antifungal agent.

22. A method of treating a subject for an infection by bacteria or fungi in a growth arrest phase, the method comprising administering a therapeutically effective amount of the composition any one of claims 12-21 to the subject.

23. The method of claims 22, wherein the composition is administered locally at the site of infected tissue.

24. The method of claim 23, wherein the infection is an ear infection, and the composition is administered locally into the ear canal.

25. The method of claim 22, wherein the infection is a chronic bacterial or fungal infection.

26. The method of claim 25, wherein the infection is tuberculosis, cystic fibrosis, a cutaneous wound infection, a urinary tract infection, or a biofilm-associated infection.

27. The method of claim 26, wherein the biofilm-associated infection is a catheter associated infection, a central line-associated infection, an endotracheal tube associated infection, an implantable device-associated infection, or a prosthetic joint-associated infection.

28. The method of any one of claims 22-27, further comprising administering a therapeutically effective amount of at least one antibiotic or antifungal agent to the subject.

29. The method of any one of claims 22-28, wherein multiple cycles of treatment are administered to the subject.

30. The method of any one of claims 22-29, wherein the bacteria are Gram-negative bacteria.

31. A composition for use in a method of treating cancer, the composition comprising a metallic nanocluster having a size of less than 10 nm, wherein the nanocluster is conjugated to adenosine triphosphate (ATP) or an analogue thereof.

32. The composition of claim 31, further comprising a pharmaceutically acceptable excipient or carrier.

33. The composition of claim 31 or 32, further comprising an anti-cancer agent.

34. The composition of any one of claims 31-33, wherein the metallic nanocluster comprises a noble metal.

35. The composition of claim 34, wherein the noble metal is gold.

36. The composition of any one of claims 31-35, wherein the nanocluster is conjugated to at least 1000 ATP molecules.

37. The composition of any one of claims 37-39, wherein the cancer is melanoma or schwannoma.

38. A method of treating cancer in a subject, the method comprising administering a therapeutically effective amount of the composition of any one of claims 31-37 to the subject.

39. The method of claim 38, wherein the composition is administered locally, intratumorally, intravenously, subcutaneously, by inhalation, or topically.

40. The method of claim 38, wherein the composition is administered locally to a tumor.

41. The method of any one of claims 38-40, wherein multiple cycles of treatment are administered to the subject.

42. The method of any one of claims 38-41, further comprising administering a therapeutically effective amount of ATP to the subject.

43. The method of any one of claims 38-42, wherein the cancer is melanoma or schwannoma.

44. The method of any one of claims 38-43, wherein the nanocluster has a diameter of less than 5 nm.

45. The method of claim 44, wherein the diameter ranges from about 1 nm to about 5 nm.

46. The method of claim 45, wherein the diameter is about 2 nm.

47. The method of any one of claims 38-46, wherein the ATP analogue is selected from the group consisting of ATPαS, ATPβS, ATPγS, deoxyadenosine triphosphate (dATP), 7-deazaadenosine-5′-triphosphate (7-deaza-ATP, and β,γ-methyleneadenosine 5′-triphosphate (AMP-PCP).

48. The method nanocluster of any one of claims 38-47, wherein the metallic nanocluster comprises a noble metal.

49. The method of claim 48, wherein the noble metal is gold.

50. The method of any one of claims 38-49, wherein the nanocluster is conjugated to at least 1000 ATP molecules.

51. A method of treating melanoma in a subject, the method comprising administering to the subject a therapeutically effective amount of ATP in combination with a therapeutically effective amount of a metallic nanocluster having a size of less than 10 nm, wherein the nanocluster is conjugated to adenosine triphosphate (ATP) or an analogue thereof.

52. The method of claim 51, wherein the ATP and the metallic nanocluster are administered intratumorally or topically.

53. The method of claim 51 or 52, wherein the nanocluster has a diameter of less than 5 nm.

54. The method of anyone of claims 51-53, wherein the diameter ranges from about 1 nm to about 5 nm.

55. The method of claim 54, wherein the diameter is about 2 nm.

56. The method of any one of claims 51-55, wherein the ATP analogue is selected from the group consisting of ATPαS, ATPβS, ATPγS, deoxyadenosine triphosphate (dATP), 7-deazaadenosine-5′-triphosphate (7-deaza-ATP, and β,γ-methyleneadenosine 5′-triphosphate (AMP-PCP).

57. The method nanocluster of any one of claims 51-56, wherein the metallic nanocluster comprises a noble metal.

58. The method of claim 57, wherein the noble metal is gold.

59. The method of any one of claims 51-58, wherein the nanocluster is conjugated to at least 1000 ATP molecules.

60. A method of inhibiting a FtsH protease, the method comprising contacting the FtsH protease with a metallic nanocluster having a size of less than 10 nm, wherein the nanocluster is conjugated to adenosine triphosphate (ATP) or an analogue thereof, wherein the protease activity of the FtsH protease is inhibited.

61. The method of claim 60, wherein the nanocluster has a diameter of less than 5 nm.

62. The method of claim 61, wherein the diameter ranges from about 1 nm to about 5 nm.

63. The method of claim 62, wherein the diameter is about 2 nm.

64. The method of any one of claims 60-63, wherein the ATP analogue is selected from the group consisting of ATPαS, ATPβS, ATPγS, deoxyadenosine triphosphate (dATP), 7-deazaadenosine-5′-triphosphate (7-deaza-ATP, and β,γ-methyleneadenosine 5′-triphosphate (AMP-PCP).

65. The method nanocluster of any one of claims 60-64, wherein the metallic nanocluster comprises a noble metal.

67. The method of claim 65, wherein the noble metal is gold.

66. The method of any one of claims 60-67, wherein the nanocluster is conjugated to at least 1000 ATP molecules.

68. A method of inhibiting a purinergic P2X7 receptor (P2X7R), the method comprising contacting the P2X7R with a metallic nanocluster having a size of less than 10 nm, wherein the nanocluster is conjugated to adenosine triphosphate (ATP) or an analogue thereof, wherein the activity of the P2X7R is inhibited.

69. A method of increasing phagocytic clearance in a tissue, the method comprising contacting the tissue with a metallic nanocluster having a size of less than 10 nm, wherein the nanocluster is conjugated to adenosine triphosphate (ATP) or an analogue thereof, wherein the phagocytic clearance is increased in the tissue.

70. A method of reducing NLRP3 activation and IL-1beta-mediated inflammation in a subject, the method comprising administering a therapeutically effective amount of a metallic nanocluster having a size of less than 10 nm to the subject, wherein the nanocluster is conjugated to adenosine triphosphate (ATP) or an analogue thereof.

71. The method of claim 70, wherein said administering the therapeutically effective amount of the metallic nanocluster reduces microglial inflammation, reduces oxidative stress, and increases phagocytic clearance in the subject.

72. The method of claim 70 or 71, wherein the nanocluster has a diameter of less than 5 nm.

73. The method of claim 72, wherein the diameter ranges from about 1 nm to about 5 nm.

74. The method of claim 73, wherein the diameter is about 2 nm.

75. The method of any one of claims 70-74, wherein the ATP analogue is selected from the group consisting of ATPαS, ATPβS, ATPγS, deoxyadenosine triphosphate (dATP), 7-deazaadenosine-5′-triphosphate (7-deaza-ATP, and β,γ-methyleneadenosine 5′-triphosphate (AMP-PCP).

76. The method nanocluster of any one of claims 70-75, wherein the metallic nanocluster comprises a noble metal.

77. The method of claim 76, wherein the noble metal is gold.

78. The method of any one of claims 70-77, wherein the nanocluster is conjugated to at least 1000 ATP molecules.

79. A method of inducing endoplasmic reticulum (ER) stress in a cell, the method comprising contacting the cell with an effective amount of a metallic nanocluster having a size of less than 10 nm, wherein the nanocluster is conjugated to adenosine triphosphate (ATP) or an analogue thereof.

80. The method of claim 79, wherein the cell is a cancerous cell.

81. The method of claim 79 or 80, wherein the nanocluster has a diameter of less than 5 nm.

82. The method of claim 81, wherein the diameter ranges from about 1 nm to about 5 nm.

83. The method of claim 82, wherein the diameter is about 2 nm.

84. The method of any one of claims 79-83, wherein the ATP analogue is selected from the group consisting of ATPαS, ATPβS, ATPγS, deoxyadenosine triphosphate (dATP), 7-deazaadenosine-5′-triphosphate (7-deaza-ATP, and β,γ-methyleneadenosine 5′-triphosphate (AMP-PCP).

85. The method nanocluster of any one of claims 79-84, wherein the metallic nanocluster comprises a noble metal.

86. The method of claim 85, wherein the noble metal is gold.

87. The method of any one of claims 79-86, wherein the nanocluster is conjugated to at least 1000 ATP molecules.

88. A method of inhibiting proliferation of a cancerous cell, the method comprising contacting the cell with an effective amount of a metallic nanocluster having a size of less than 10 nm, wherein the nanocluster is conjugated to adenosine triphosphate (ATP) or an analogue thereof.

89. The method of claim 88, wherein the cancerous cell is a melanoma or schwannoma cell.

90. The method of claim 88 or 89, wherein the nanocluster has a diameter of less than 5 nm.

91. The method of claim 90, wherein the diameter ranges from about 1 nm to about 5 nm.

92. The method of claim 91, wherein the diameter is about 2 nm.

93. The method of any one of claims 88-92, wherein the ATP analogue is selected from the group consisting of ATPαS, ATPβS, ATPγS, deoxyadenosine triphosphate (dATP), 7-deazaadenosine-5′-triphosphate (7-deaza-ATP, and β,γ-methyleneadenosine 5′-triphosphate (AMP-PCP).

94. The method nanocluster of any one of claims 88-93, wherein the metallic nanocluster comprises a noble metal.

95. The method of claim 94, wherein the noble metal is gold.

96. The method of any one of claims 88-95, wherein the nanocluster is conjugated to at least 1000 ATP molecules.
